SEQUENCE LISTING (26 sequences)

SEQ ID NO: 1 - protein, 114 aa, Homo sapiens
QAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCTCR

SEQ ID NO: 2 - protein, 183 aa, Homo sapiens
STGVLLPLQNNELPGAEYQYKKDEVWEERKPYKTLQAQAPEKSKNKKKQRKGPHRKSQTLQFDEQTLKKARRKQWIEPRNCARRYLKVDFADIGWSEWIISPKSFDAYYCSGACQFPMPKSLKPSNHATIQSIVRAVGVVPGIPEPCCVPEKMSSLSILFFDENKNVVLKVYPNMTVESCACR

SEQ ID NO: 3 - protein, 116 aa, Homo sapiens
SPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 4 - protein, 138 aa, Homo sapiens
AANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 5 - protein, 139 aa, Homo sapiens
SASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 6 - protein, 139 aa, Homo sapiens
STGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGGH

SEQ ID NO: 7 - protein, 133 aa, Homo sapiens
RRQPKKSNELPQANRLPGIFDDVHGSHGRQVCRRHELYVSFQDLGWLDWVIAPQGYSAYYCEGECSFPLDSCMNATNHAILQSLVHLMMPDAVPKACCAPTKLSATSVLYYDSSNNVILRKHRNMVVKACGCH

SEQ ID NO: 8 - protein, 110 aa, Homo sapiens
SAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 9 - protein, 108 aa, Homo sapiens
NAKGNYCKRTPLYIDFKEIGWDSWIIAPPGYEAYECRGVCNYPLAEHLTPTKHAIIQALVHLKNSQKASKACCVPTKLEPISILYLDKGVVTYKFKYEGMAVSECGCR

SEQ ID NO: 10 - protein, 108 aa, Homo sapiens; identical to SEQ ID NO: 9

SEQ ID NO: 11 - protein, 104 aa, Homo sapiens
SRCSRKPLHVDFKELGWDDWIIAPLDYEAYHCEGLCDFPLRSHLEPTNHAIIQTLLNSMAPDAAPASCCVPARLSPISILYIDAANNVVYKQYEDMVVEACGCR

SEQ ID NO: 12 - protein, 120 aa, Homo sapiens
TAFASRHGKRHGKKSRLRCSKKPLHVNFKELGWDDWIIAPLEYEAYHCEGVCDFPLRSHLEPTNHAIIQTLMNSMDPGSTPPSCCVPTKLTPISILYIDAGNNVVTKQYEDMVVESCGCR

SEQ ID NO: 13 - protein, 120 aa, Homo sapiens
APLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 14 - protein, 281 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAGHANHGFVVEVAHLEEKQGVSKRHVRISRSLHQDEHSWSQIRPLLVTFGHDGKGHPLHKREKR

SEQ ID NO: 15 - protein, 289 aa, Homo sapiens
MAGASRLLFLWLGCFCVSLAQGERPKPPFPELRKAVPGDRTAGGGPDSELQPQDKVSEHMLRLYDRYSTVQAARTPGSLEGGSQPWRPRLLREGNTVRSFRAAAAETLERKGLYIFNLTSLTKSENILSATLYFCIGELGNISLSCPVSGGCSHHAQRKHIQIDLSAWTLKFSRNQSQLLGHLSVDMAKSHRDIMSWLSKDITQFLRKAKENEEFLIGFNITSKGRQLPKRRLPFPEPYILVYANDAAISEPESVVSSLQGHRNFPTGTVPKWDSHIRAALSIERRKKR

SEQ ID NO: 16 - protein, 292 aa, Homo sapiens
MIPGNRMLMVVLLCQVLLGGASHASLIPETGKKKVAGIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEAISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKR

SEQ ID NO: 17 - protein, 316 aa, Homo sapiens
MHLTVFLLKGIVGFLWSCWVLVGYAKGGLGDNHVHSSFIYRRLRNHERREIQREILSILGLPHRPRPFSPGKQASSAPLFMLDLYNAMTNEENPEESEYSVRASLAEETRGARKGYPASPNGYPRRIQLSRTTPLTTQSPPLASLHDTNFLNDADMVMSFVNLVERDLDFSHQRRHYKEFRFDLTQIPHGEAVTAAEFRIYKDRSNNRFENETIKISIYQIIKEYTNRDADLFLLDTRKAQALDVGWLVFDITVTSNHWVINPQNNLGLQLCAETGDGRSINVKSAGLVGRQGPQSKQPFMVAFFKASEVLLRSVR

SEQ ID NO: 18 - protein, 374 aa, Homo sapiens
MPGLGRRAQWLCWWWGLLCSCCGPPPLRPPLPAAAAAAAGGQLLGDGGSPGRTEQPPPSPQSSSGFLYRRLKTQEKRGMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGIVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTR

SEQ ID NO: 19 - protein, 292 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIR

SEQ ID NO: 20 - protein, 269 aa, Homo sapiens
MTALPGPLWLLGLALCALGGGGPGLRPPPGCPQRRLGARERRDVQREILAVLGLPGRPRPRAPPAASRLPASAPLFMLDLYHAMAGDDDEDGAPAERRLGRADLVMSFVNMVERDRALGHQEPHWKEFRFDLTQIPAGEAVTAAEFRIYKVPSIHLLNRTLHVSMFQVVQEQSNRESDLFFLDLQTLRAGDEGWLVLDVTAASDCWLLKRHKDLGLRLYVETEDGHSVDPGLAGLLGQRAPRSQQPFVVTFFRASPSPIRTPRAVRPLR

SEQ ID NO: 21 - protein, 318 aa, Homo sapiens
MCPGALWVALPLLSLLAGSLQGKPLQSWGRGSAGGNAHSPLGVPGGGLPEHTFNLKMFLENVKVDFLRSLNLSGVPSQDKTRVEPPQYMIDLYNRYTSDKSTPASNIVRSFSMEDAISITATEDFPFQKHILLFNISIPRHEQITRAELRLYVSCQNHVDPSHDLKGSVVIYDVLDGTDAWDSATETKTFLVSQDIQDEGWETLEVSSAVKRWVRSDSTKSKNKLEVTVESHRKGCDTLDISVPPGSRNLPFFVVFSNDHSSGTKETRLELREMISHEQESVLKKLSKDGSTEAGESSHEEDTDGHVAAGSTLARRKR

SEQ ID NO: 22 - protein, 316 aa, Homo sapiens
MGSLVLTLCALFCLAAYLVSGSPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPELDNLGLDSFSSGPGEEALLQMRSNIIYDSTARIRR

SEQ ID NO: 23 - protein, 316 aa, Homo sapiens
MGSLVLTLCALFCLAAYLVSGSPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPEEDNLGLDSFSSGPGEEALLQMRSNIIYDSTARIRR

SEQ ID NO: 24 - protein, 346 aa, Homo sapiens
MDLSAAAALCLWLLSACRPRDGLEAAAVLRAAGAGPVRSPGGGGGGGGGGRTLAQAAGAAAVPAAAVPRARAARRAAGSGFRNGSVVPHHFMMSLYRSLAGRAPAGAAAVSASGHGRADTITGFTDQATQDESAAETGQSFLFDVSSLNDADEVVGAELRVLRRGSPESGPGSWTSPPLLLLSTCPGAARAPRLLYSRAAEPLVGQRWEAFDVADAMRRHRREPRPPRAFCLLLRAVAGPVPSPLALRRLGFGWPGGGGSAAEERAVLVVSSRTQRKESLFREIRAQARALGAALASEPLPDPGTGTASPRAVIGGRRRRRTALAGTRTAQGSGGGAGRGHGRRGR

SEQ ID NO: 25 - protein, 335 aa, Homo sapiens
MDTPRVLLSAVFLISFLWDLPGFQQASISSSSSSAELGSTKGMRSRKEGKMQRAPRDSDAGREGQEPQPRPQDEPRAQQPRAQEPPGRGPRVVPHEYMLSIYRTYSIAEKLGINASFFQSSKSANTITSFVDRGLDDLSHTPLRRQKYLFDVSMLSDKEELVGAELRLFRQAPSAPWGPPAGPLHVQLFPCLSPLLLDARTLDPQGAPPAGWEBFDVWQGLRHQPWKQLCLELRAAWGELDAGEAEARARGPQQPPPPDLRSLGFGRRVRPPQERALLVVFTRSQRKNLFAEMREQLGSAEAAGPGAGAGGSWPPPSGAPDARPWLPSPGRRRRR

SEQ ID NO: 26 - protein, 381 aa, Homo sapiens
MRLPKLLTFLLWYLAWLDLEFICTVLGAPDLGQRPQGTRPGLAKAEAKERPPLARNVFRPGGHSYGGGATNANARAKGGTGQTGGLTQPKKDEPKKLPPRPGGPEPKPGHPPQTRQATARTVTPKGQLPGGKAPPKAGSVPSSFLLKKAREPGPPREPKEPFRPPPITPHEYMLSLYRTLSDADRKGGNSSVKLEAGLANTITSFIDKGQDDRGPVVRKQRYVFDISALEKDGLLGAELRILRKKPSDTAKPAAPGGGRAAQLKLSSCPSGRQPASLLDVRSVPGLDGSGWEVFDIWKLFRNFKNSAQLCLELEAWERGRAVDLRGLGFDRAARQVKEKALFLVFGRTKKRDLFFNEIKARSGQDDKTVYEYLFSQRRKRR

(19) United States
(12) Patent Application Publication
(10) Pub. No.: US 2007/0105762 A1
(43) Pub. Date: May 10, 2007

(54) Non-activated polypeptides having a function of tissue regeneration and method for preparing the same

(75) Inventors: Jung Moon Kim, Seoul (KR); Jung Moon Kim, Seoul (KR); Tae Han Kim, Gyeonggi-do (KR); Jong Suk Lee, Seoul (KR); Jong In Yook, Seoul (KR)

Correspondence Address: INTELLECTUAL PROPERTY / TECHNOLOGY LAW, PO BOX 14329, RESEARCH TRIANGLE PARK, NC, 27709, US

(73) Assignees: Jung Moon Kim, Seoul (KR); Jung Kook Kim, Seoul (KR); Tae Han Kim, Gyeonggi-Do (KR); Jong Suk Lee, Seoul (KR)

(21) Appl. No.: 10/560,329

(22) PCT Filed: Nov. 2, 2005
(86) PCT No.: PCT/KR05/03660

§ 371 Date: Dec. 10, 2005

(30) Foreign Application Priority Data
Mar. 30, 2005 (KR) 10-2005-0026246
Oct. 31, 2005 (KR) 10-2005-0103040

(51) Int. Cl. A61K 38/17; C07K 14/475; C07H 21/04; C12P 21/06

(52) U.S. Cl. 514/12; 530/350; 435/69.1; 435/320.1; 435/325; 536/23.5

(57) ABSTRACT

Non-activated tissue-regeneration polypeptides (TRPs) and the preparation methods thereof are disclosed. The TRPs contain: a protein transduction domain (PTD) making the polypeptides to permeate a cell membrane without cell membrane receptors; a furin activation domain (FAD) which has at least one proprotein convertase cleavage site and which can be cleaved by the proprotein convertase and activate a tissue regeneration domain (TRD) in cells; and a tissue regeneration domain (TRD) which can be activated by the proprotein convertase cleavage of the FAD to stimulate the growth or formation of tissues or to induce the regeneration of tissues. The TRPs can be practically mass-produced by the culture of bacteria, such as recombinant E. coli, and are in a non-activated state before in vivo administration. Thus, their production cost is only a few tenths of the prior active proteins having uses similar thereto, and processes for their separation, purification, handling, storage and administration are significantly simple and convenient. The in vivo administration of the TRPs can stimulate the formation or regeneration of tissues, such as bones or cartilages, or improve the fibrosis and cirrhosis of organs, such as kidneys, liver, lungs and heart by pharmacological mechanisims completely different from those of prior rhBMPs or TGF-β proteins. Accordingly, the TRPs will be useful as drugs having new mechanisms.

### TECHNICAL FIELD

The present invention is related to non-activated tissue-regenerative polypeptides (TRPs) and the methods for preparing the TRPs, the tissue regenerative polypeptides, which contain: a protein transduction domain (PTD) making the polypeptides to permeate a cell membrane without cell membrane receptors; a furin activation domain (FAD) which has at least one proprotein convertase cleavage site and which can be cleaved by the proprotein convertase and can activate a non-activated tissue regeneration domain (TRD) in cells; and a non-activated tissue regeneration domain (TRD) which can be activated through the the cleavage of the FAD by the proprotein convertase in cells and which can stimulate the growth or formation of tissues or to induce tissue regeneration.

### BACKGROUND ART

It is known that bone morphogenetic proteins (BMPs) stimulate the healing of bone defects in mammals or Primates, and particularly are secreted in bone cells to induce bone formation through receptors present in the adjacent cell membranes, when used with collagen or biodegradable polymers in demineralization conditions. The hBMPs are a group of proteins having similarity to each other, and are known to have more than 14 members, including hBMP2 through hBMP15 until now. The hBMP2, 3, 4, 6, 7 and 14 are known to have the medical efficacy of inducing bone regeneration.

Among them, the hBMP2 has been known to be the most effective bone morphogenetic protein. There have been a lot of studies on the medical effects and applications of BMP proteins. The BMP7 is known to not only suppress the fibrosis of organs by antagonistic action of TGF-β1, but also induce the regeneration of organs (Nature Med., 9:964, 2003; J. Biol. Chem., 280:8094, 2005). It is known that the BMP14, (GDF-5, growth/differentiation factor-5: MP-52) shows the function of effectively healing skin wounds and aiding the healing of gastric or duodenal ulcer when administered to human beings or mammals (U.S. Pat. No. 6,764,994; Nature, 368:639, 1994; Exp. Cell Res., 235:218, 1997; Neurosurg Focus, 13:1, 2002; U.S. Pat. No. 6,531,450). In the case of liver or kidney cirrhosis, the BMPs are also known to antagonize TGF-β to inhibit the formation of fibrous tissue and induce the recovery of normal tissue. Namely, the BMPs may function not only to stimulate the formation of bones or cartilages but also to regenerate the skin or regenerate gastrointestinal tissue, unlike the terminological definition of BMP.

As an old method for preparing BMPs, a method of extracting BMPs from demineralized animal bone tissue using natural salt (U.S. Pat. No. 4,294,753) has been attempted, but the method has problems in that the preparation efficiency is too low for mass production. A method was developed in early 1990s for separating and purifying the active recombinant hBMP2 after culturing CHO cells transformed with a BMP gene (Proc. Natl. Acad. Sci., 87:2220, 1990). Since then, the mass production of BMPs has been possible (U.S. Pat. No. 4,968,590; 5,106,626; 5,106,748; 5,166,058; 5,187,076; 5,187,623; 5,208,219; 5,258,494; 5,266,683; 5,284,756; 5,399,346; and 6,593,109).

However, the methods for preparing recombinant hBMP2 and hBMP7 using the transformed CHO cells have problems in that the culturing of a large amount of CHO cells is required to obtain enough amount of active rhBMP2 or rhBMP7. The separation and purification steps are very complicated, and the production cost is very high. Also, these methods have a common problem that the biological activity of the proteins is reduced during the separation, purification, storage, medication and/or administration processes. To improve a part of the shortcomings of these methods of preparing recombinant BMPs by culturing animal cells, Biopharm company recently developed a method for preparing rhBMP14(MP-52) by culturing recombinant E. coli at lower cost (U.S. Pat. No. 2003/0181378; WO 96/33215). In this method of preparing the rhBMP14, however, the step of separating and purifying rhBMP14 in the form of an activated protein is still complicated and inconvenient. The problem of activity reduction of the prepared E. coli recombinant protein has still not been solved for the separation, purification, storage, handling, and/or administration steps.

Moreover, in producing active BMPs in recombinant E. coli as in the case of the prior Biopharm's method, there is a limitation on the selection and designing of the biochemical structures of BMPs. Even if it is possible to prepare a protein having the structure like rhBMP14, using the Biopharm's method, it shall not be possible to practically prepare the BMPs having similar biochemical structures like active rhBMP2, rhBMP4, rhBMP7, etc.

In the case of rhBMP-2, which is commercially available and is medically used for, e.g., spine fusion, it is being sold for as high as several thousand US dollars per mg in the year 2005. Despite that the rhBMPs have various latent potencies for numerous patients who need spine fusion operations, or who need regenerating gastric ulcer or liver cirrhosis, the clinical application of BMPs has been limited because of the extremely high cost and the inconveniencies and activity loss in the storage, handling and administration.

Accordingly, in the art, there has been an urgent and strong need for the development of new kinds of biochemical drug substances which would provide equal or higher biomedical efficacy than those of rhBMPs or TGF-βs, and which can be produced at significantly lower costs than those for previously known proteins, and which can fundamentally solve the inconveniences and the activity reduction problem in the separation, purification, storage, handling, and administration steps.

Accordingly, the present inventors have spent extensive time and efforts to develop a group of new polypeptides, which can fundamentally solve the problems in the previously known activated proteins produced for stimulating the formation of bones or cartilages, or the regeneration of biological tissues including the skin wounds, ulcer, and/or liver cirrhosis. The new polypeptides have new biochemical structures and provide biomedical efficacy by new pharmacological mechanisms. The new kind of polypeptides can be directly medicated to human patients to stimulate the formation or regeneration of bones or cartilages or to improve or regenerate the fibrosis or cirrhosis of organs, such as kidneys, liver, lungs and heart, by new pharmacological mechanisms. The newly developed polypeptides are kept in a non-activated state in steps of the separation, purification, storage, and handling before administration. The new polypeptides are designed so that they shall be activated in vivo after administration to patients.

Namely, the present inventors have developed a new group of non-activated tissue-regeneration polypeptides (TRPs) containing: a protein transduction domain (PTD) making the proteins to permeate cell membranes; a furin activation domain (FAD) which has at least one proprotein convertase cleavage site and which can be cleaved by the proprotein convertase and can activate a non-activated tissue regeneration domain (TRD) in cells; and a non-activated TRD which can be activated by the proprotein convertase cleavage of the FAD. The in vivo activated polypeptides are designed to stimulate the growth or formation of tissue or to induce the regeneration of tissue. Also, the present inventors have found that the production cost of the present TRPs are significantly lower than those of the existing active proteins such as rhBMPs. It is much easier to store, handle, and administer the present TRPs than the existing rhBMPs. The present TRPs provide biomedical efficacies by new pharmacological mechanisms. Specifically the TRPs permeate cell membranes without help from receptors. They are cleaved by furin in cells and then activated. The activated proteins are secreted out of the cells in large amount, and then they stimulate cell membrane receptors for bone formation or tissue regeneration. On the basis of these new findings, the present invention has been completed.

### SUMMARY OF THE INVENTION

A primary object of the present invention is to provide non-activated TRPs having biochemical structures, characteristics and pharmacological mechanisms that are completely different from the previously known active rhBMP proteins. The new non-activated TRPs are designed so that they can be administered directly into the human body to stimulate the formation or regeneration of bones and cartilages or to improve the fibrosis and cirrhosis of organs, such as kidneys, liver, lungs and heart, and ultimately to induce tissue regeneration. The present invention also provides practical production and preparation methods for the TRPs.

Another object of the present invention is to provide a new drug composition for stimulating the formation or regeneration of tissues, such as bones and cartilages, or a new drug composition for improving the fibrosis or cirrhosis of organs, such as kidneys, liver, lungs and heart, the composition containing said non-activated TRPs.

To achieve the above objects, in one aspect, the present invention provides a non-activated tissue-regeneration polypeptides (TRPs) containing:
(a) a protein transduction domain (PTD) making the polypeptides to permeate cell membranes without cell membrane receptors;
(b) a furin activation domain (FAD) which has at least one proprotein convertase cleavage site and which can be cleaved by the proprotein convertase and can activate a non-activated tissue regeneration domain (TRD) in cells; and
(c) a non-activated TRD which can be activated in vivo by the proprotein convertase cleavage of the FAD. The in vivo activated proteins are designed to stimulate the growth or formation of tissues or to induce the regeneration of tissues.

In another aspect, the present invention provides a recombinant vector inserted with an FAD-encoding base sequence in front of the 5′ region of TRD-encoding DNA, a PTD base sequence, a base sequence for tagging, and at least four histidine-encoding base sequences for separation and purification. The present invention also provides bacteria transformed with the said recombinant vectors.

In still another aspect, the present invention provides a method for preparing non-activated TRPs, comprising the steps of:
(a) culturing the said transformed bacteria to express a [PTD-FAD-TRD] polypeptide; and
(b) centrifuging the culture broth, and then removing the two-dimensional or three-dimensional structure of the polypeptides or converting the two-dimensional or three-dimensional structure to one-dimensional linear structure by addition of a urea solution into the supernatant and cell pellet, and then purifying the [PTD-FAD-TRD] polypeptide..

In the present invention, the proprotein convertase may preferably be furin but is not limited thereto. Examples thereof include PC7, PC5/6A, PC5/6B, PACE4, PC 1/3, PC2 and PC4.

Also, the TRD may preferably be represented by an amino acid sequence selected from the group consisting of SEQ ID NOs: 1 to 13, but is not specifically limited if it is a secreted protein showing the activity to stimulate the growth or formation of tissue or to induce tissue regeneration following the furin cleavage. Examples thereof include polypeptides such as BMPs, TGF-β, β-NGF (β-nerve growth factor), β-amyloid, ADAMs (a disintergrin and metalloproteinase-like), TNF-α, MMPs (matrix metalloproteinases), and insulin-like growth factor (IGF-1).

The FAD may preferably be represented by an amino acid sequence selected from the group consisting of SEQ ID NOs: 14 to 26, but is not specifically limited if it has a proprotein convertase cleavage site and if it can be cleaved by the proprotein convertase in cells to activate the TRD. Also, the PTD may preferably be selected from the group consisting of TAT, drosophila melanogaster-derived Antp peptide, VP22 peptide and mph-1-btm, but is not limited thereto.

In the inventive method for preparing the non-activated TRPs, the purification step may preferably comprise the sub-steps of binding the polypeptides to nickel-titanium beads, washing the beads with the same solution, and then eluting the beads with imidazole and a high-salt buffer solution, but is not limited thereto.

The non-activated TRPs according to the present invention have no three-dimensional stereoregularity that are possessed in common by the previously known active BMPs. The TRPs are not biologically active by themselves yet before administered into patients. When the non-activated TRPs are administered to the human beings or mammals, however, the proprotein convertase cleavage sites of FAD in the TRPs are cleaved by proprotein convertase present in living cells, whereby TRD is activated, and the activated TRD is secreted out of the cells, thereby showing the desired tissue regeneration potencies. The TRPs according to the present invention are preferably in the form of a fusion polypeptide of PTD, FAD and TRD. The inventive TRPs have a function to stimulate the formation or regeneration of tissues, such as human bones and cartilages, or to improve the fibrosis or cirrhosis of organs, such as kidneys, liver, lungs and heart, and ultimately to induce the regeneration of original tissue.

Accordingly, the present invention provides a new drug composition which contains the non-activated TRP as an active ingredient and which stimulates the formation or regeneration of tissue by new pharmacological mechanisms completely different from those of previously known protein medicines having uses similar thereto. In the present invention, the tissues may preferably be bone or cartilage. Furthermore, the present invention provides a new drug composition for improving the fibrosis or cirrhosis of organs, which contains the non-activated TRP as an active ingredient. The inventive composition may suitably contain, in addition to TRP, other growth factors, such as TGF-β (transforming growth factor-β), IGF (insulin-like growth factor), PDGF (platelet-derived growth factor), and FGF (fibroblast growth factor), in which case the therapeutic effect of the composition can be significantly increased.

Other features and embodiments of the present invention will be more clearly understood from the following detailed description and accompanying claims.

### BRIEF DESCRIPTION OF DRAWINGS

FIG. 1 shows a structural schematic diagram of human BMP2, BMP7 and TGF-β proteins and the total amino acid sequence thereof.

FIG. 2 is a photograph showing the two-dimensional and three-dimensional structures of previously commercially available rhBMP-2.

FIG. 3 shows a process for the preparation, separation and purification of TRP-1 according to the present invention.

FIG. 4 shows a process for the preparation, separation and purification of the previously known rhBMP.

FIG. 5 shows the pharmacological mechanism of the previous rhBMP.

FIG. 6 shows the pharmacological mechanisms of the non-activated TRPs prepared according to the present invention.

FIG. 7 shows a recombinant expression vector having TAT bound to an hBMP2 gene.

FIG. 8 shows electrophoresis photographs of a total protein before and after inducing protein expression by IPTG during the culture of E. coli transformed with the recombinant expression vector shown in FIG. 7.

FIG. 9 shows the results of Western blot analysis for TAT-BMP2 using an anti-X-press antibody.

FIG. 10 is a Western blot photograph showing that TAT-BMP2 permeates cell membranes to transduce into the cells.

FIG. 11 is a photograph showing the alkaline phosphatase (ALP) activity of TAT-BMP2.

FIG. 12 is a photograph showing that fibroblasts treated with TAT-BMP2 and commercially available rhBMP-2 were subjected to von Kossa staining to examine the differentiation of bone cells and the formation of mineralized substances.

FIG. 13 is a schematic diagram of TRP-1 variant designed to examine a furin activation mechanism, which shows that a signal peptide, expected to regulate the post-translational modification, and a pro-domain, activated by furin, are bound to BMP2.

FIG. 14 is a Western blot photograph showing that the inventive TRP-1 and its variant permeate cell membranes to transduce into the cells.

FIG. 15 is a photograph showing that the inventive TRP-1 is cleaved by furin in vitro.

FIG. 16 shows the results of Western blot analysis using α1-PDX and anti-X-press, which indicate that the inventive TRP- 1 is activated by furin in cells.

FIG. 17 shows measurement results for ALP activity in fibroblasts treated with the inventive TRP- 1 and its variants.

FIG. 18 shows that fibroblasts treated with the inventive TRP-1 and its variants were subjected to von Kossa staining to examine the differentiation of bone cells and the formation of mineralized substances.

FIG. 19 shows electrophoresis photographs of a total protein before and after inducing TRP- 1 and TRP-2 expressions by IPTG during the culture of the inventive recombinant E. coli.

FIG. 20 shows the results of Western blot analysis for the inventive TRP-1 and TRP-2 using an anti-X-press antibody.

FIG. 21 illustrates the results of Western blot analysis using an anti-X-press antibody, which show the process of the inventive TRP-1 being introduced into cells.

FIG. 22 illustrates the results of Western blot analysis using an anti-X-press antibody, which show the process of the inventive TRP-2 being introduced into cells.

FIG. 23 illustrates the results for Western blot analysis conducted to examine the secretion of activated hBMP2 in fibroblasts treated with the inventive TRP- 1.

FIG. 24 shows that fibroblasts treated with the inventive TRP-1 and commercially available rhBMP-2 were subjected to von Kossa staining to examine the differentiation of bone cells and the formation of mineralized substances.

FIG. 25 shows measurement results for ALP activity in fibroblasts treated with the inventive TRP-1 and commercially available rhBMP-2.

FIG. 26 shows that fibroblasts treated with the TRP-1 and rhBMP-2 at various concentrations were subjected to von Kossa staining to examine the differentiation of bone cells and the formation of mineralized substances.

FIG. 27 shows the results of Western blot analysis using an anti-X-press antibody, which indicate the half-life of the inventive TRP- 1 introduced into cells.

FIG. 28 is a confocal microphotograph showing observation results for the intracellular permeation and temperature effect of the inventive TRP- 1 and TRP-2.

FIG. 29 is a photograph showing that the inventive TRP-1 and TRP-2 specifically bind to F-actin after permeation into cells.

FIG. 30 is a photograph showing that the formation of bones is induced in test animals administered with the inventive TRP- 1.

FIG. 31 shows the comparison of cytotoxicity between the inventive TRP- 1 and the prior commercially available rhBMP-2.

### DETAILED DESCRIPTION OF THE INVENTION, AND PREFERRED EMBODIMENTS THEREOF

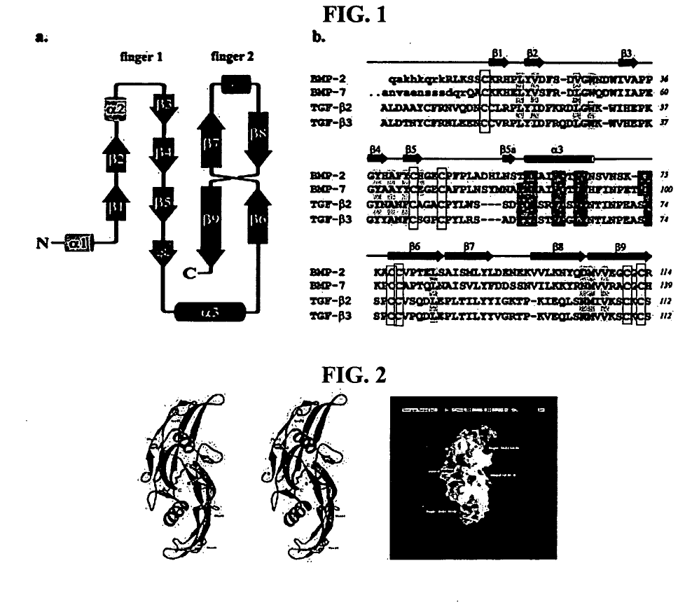

The non-activated TRPs according to the present invention have the biochemical structures and characteristics completely different from those of previously known active proteins, such as rhBMPs or TGF-β. Namely, the inventive non-activated TRPs have no biological activity by themselves before in vivo administration, whereas previously known rhBMPs and TGF-β proteins are in a biochemically activated state and have three-dimensional structures as shown in FIG. 1 and FIG. 2 (Eur. J Biochem., 237:295, 1996; J Mol. Biol., 287:103, 1999).

Thus, the preparation of prior activated rhBMPs and TGF-β has a common problem in that it should rely on the culturing of recombinant animal cells, having a significantly low productivity, such as CHO cells. As in the case of rhBMP14 (MP-52) prepared by BioPharm, some of rhBMPs are prepared by the culturing of recombinant E. coli having high productivity than the animal cells, in which case many limitations are imposed on the biochemical structure of BMP to be prepared. Namely, active MP-52 may be prepared using the recombinant E. coli, but other kinds of active BMPs, such as rhBMP2, rhBMP4 and rhBMP7 or active TGF-β can not be prepared using the recombinant E. coli.

Meanwhile, US 2004/197867 A1 discloses a fusion polypeptide of a bone morphogenetic polypeptide and a protein transduction domain (PTD), and a method for inducing bone formation in animals by administering the fusion polypeptide. More specifically, said patent discloses that administering a fusion polypeptide of inexpensive LMP-1 (LIM mineralization protein-1) and PTD instead of directly administering expensive BMP induces the mRNA expression of BMP2 and BMP7, and also said patent mentions, as bone morphogenetic proteins, not only LMP but also BMP, TGF-β, SMAD, etc. However, it is well known that, even if mRNAs are formed in cells, proteins are not formed from numerous mRNAs due to the regulation and interference of mRNAs by micro RNAs (Nature, 409:363, 2001; Cell, 115:199, 2003). Therefore the contention that the increase of BMP mRNAs by LMP-1 is a critical factor for the differentiation of bone cells lacks credibility. In addition, even if PTD is used to introduce proteins into cells, it cannot be said that all the proteins are activated to show the expected bone morphogenetic potencies (Curr. Protein Peptide Sci., 4:97, 2003). Particularly in the case of secretory proteins, such as BMPs, the pharmacological activities can be shown only through a series of complicated post-translafional modification processes, unlike other kinds of proteins showing biomedical potencies of nuclear and cytoplasmic protein in the cells.

Particularly, the BMPs work by the pharmacological mechanisms and activation mechanisms completely different from those of LMPs which is probably a nuclear transcription factor. Therefore it is not predictable at all before appropriate experimental analyses that the simple replacement of LMP-1 with BMP can show the biomedical effects similar to those obtained from LMP-TAT. If PTD is used to introduce recombinant proteins into cells as in the case of US 2004/197867 A1, LMP- 1 or SMAD may be activated to function as a gene regulatory factor, whereas BMP and TGA-β may show completely different behavior than from the case of LMP or SMAD. It is because the BMPs and TGF-β belong to secretory proteins, unlike LMPs (Genes Dev., 15:2797, 2001; J Cell Biol., 144:139, 1999; Curr. Prot. Pept. Sci., 4:97, 2003; Nature, 425:577, 2003). In other words, the BMPs and TGF-β proteins cannot show the expected biomedical potencies without highly complicated post-translational activation processes (e.g., intracellular processing and modification), unlike LMPs. Therefore it cannot be expected that, the PTD simply bound to BMPs would show the desired biological activities when it is administered in vivo. Unfortunately, the US 2004/197867 A1 does not mention any experimental evidence showing that bone formation is stimulated by the introduction of a fusion polypeptide of BMP and PTD. It does not rationalize either that the administration of BMP-PTD, instead of rhBMP, could provide any additional or differential effects than administration of prior rhBMPs.

The present inventors have found that the primary factor of the lack of efficiency in administering the previously known rhBMPs into living human beings or mammals is due to their biochemical activity and the three-dimensional steric structure. The production cost of the rhBMP becomes extremely high because of the biochemical activity of the BMPs. The low-productive animal cell culturing is preferred due to the activation of the BMPs. The separation, purification, storing, handling, and administration of the BMPs become inefficient and expensive, because the biological activity of BMPs is maintained even before administration.

Based on this finding, the present inventors first attempted to prepare a non-activated polypeptide showing no three-dimensional steroregularity at room temperature. Namely, in order to make the non-activated polypeptide to permeate cell membranes without cell membrane receptors, the non-activated polypeptides were fused with PTDs, such as TAT, and examined for permeation into cells. An inactivated BMP-TAT fusion polypeptide having no three-dimensional steric structure was prepared in this manner and was administered into cells. As shown by the Example 1 below, the fusion polypeptide of BMP-TAT, for example, easily permeate through the cell membranes, as expected, when administered into mammal cells. However, the permeated BMP-TAT does not show any biochemical activity, nor pharmacological efficacies associated with the stimulation of bone or cartilage formation or regeneration.

To additionally solve this problem, the present inventors have developed a practical method making the non-activated polypeptide to be activated, when administered into the human body, by proprotein convertase, such as furin, which is present in cells. Namely, we prepared a polypeptide (TRP) containing: PTD making the polypeptide to permeate cell membranes without cell membrane receptors of, e.g., human beings and mammals; FAD making the non-activated protein to be activated in cells by in vivo proprotein convertase, such as furin; and TRD being able to be activated to function to induce tissue growth or regeneration. The TRD can function to stimulate the formation or regeneration of bones or cartilages or improve the fibrosis and cirrhosis of kidneys, liver, lungs and heart, when permeated into animal cells by help of PTD and activated in vivo by the FAD. We have found that the above-prepared polypeptide not only permeates cell membranes but also is activated in vivo and then secreted to exhibit the effect of stimulating tissue growth or regenerating tissue.

The TRPs according to the present invention can be mass-produced in a practical manner through culturing bacteria, such as recombinant E. coli, without limitations caused by the biochemical structure of the proteins. They are maintained in a biochemically inactive state before in vivo administration. For this reason, their production cost is only a few tenths of the costs of previously known activated proteins (rhBMPs, TGF-β proteins, etc.) having uses similar thereto. The processes for separation, purification, handling, storage and administration of the present inventive TRPs are significantly simpler and more convenient than those of prior active rhBMP proteins.

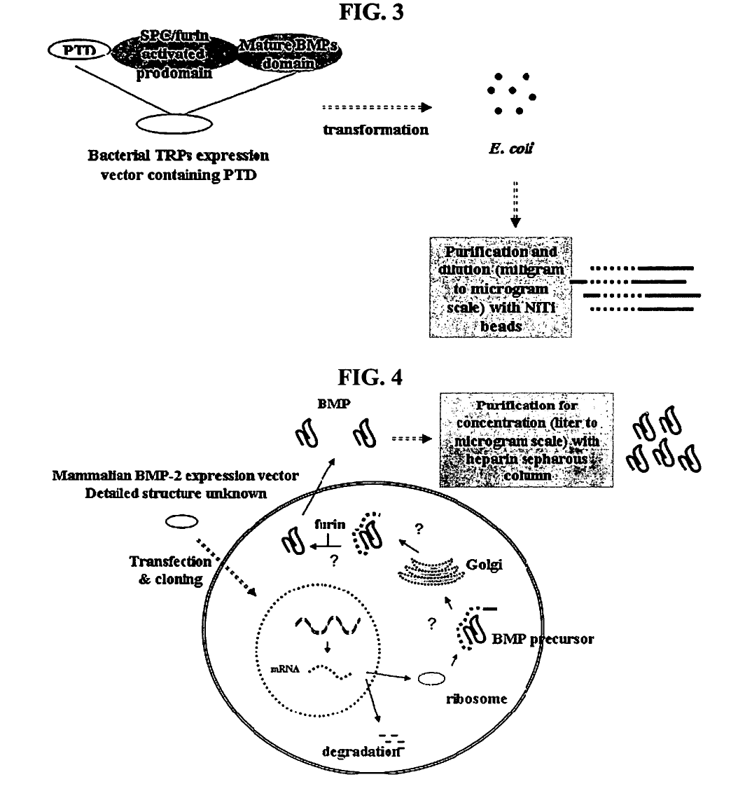

Unlike that the prior active BMPs had limitations on their preparation methods, the present invention has an advantage in that TRPs, which include BMPs and TGF-β proteins having various structures as TRDs, can be prepared using recombinant E. coli in a practical and inexpensive manner (see FIG. 3). FIG. 3 shows a process for separating and purifying TRP-1 according to the present invention, and FIG. 4 shows a process for separating and purifying rhBMPs according to the prior art (U.S. Pat. No. 4,968,590; 5,106,626; 5,106,748; 5,166,058; 5,187,076; 5,187,623; 5,208,219; 5,258,494; 5,266,683; 5,284,756; 5,399,346; and 6,593,109). As shown in the figures, the prior method includes a complicated separation and purification process and shows low purification yield because it comprises producing and secreting BMPs through the culture of transformed CHO cells. It shows very low purification yield to separate and to purify prior BMPs from a large culture medium by using herarin sepharous column, and the like. The inventive method has advantages in that it needs a very simple separation and purification process and in that it provides high purification yield. Namely TRP-1 according to the present invention is, unlike previously known rhBMP-2, in a non-activated state until administration into patients, and thus it does not require the culturing of a large amount of CHO cells. It is simple and easy to separate and purify the inventive TRP-1, and its purification yield is significantly higher than that of prior activated rhBMP-2. Accordingly, the non-activated TRPs according to the present invention have advantages in that they can be produced at the cost of a few tenths of that of the prior commercially available rhBMP-2, and in that it is convenient to store and administer the inventive TRPs.

The present invention fundamentally solves the high-cost problem possessed in common by the previously known rhBMPs, and the inefficiency problems associated with the processes of separation, purification, storage and administration of the active rhBMP products. Also, it was found that the non-activated polypeptides have new pharmacological mechanisms completely different from those of the prior rhBMPs, when administered directly into living human beings or mammals. The inventive TRPs show equal or better biomedical efficacies on the stimulation of tissue formation or the regeneration of tissue comparing with the previously known rhBMPs or TGF-β.

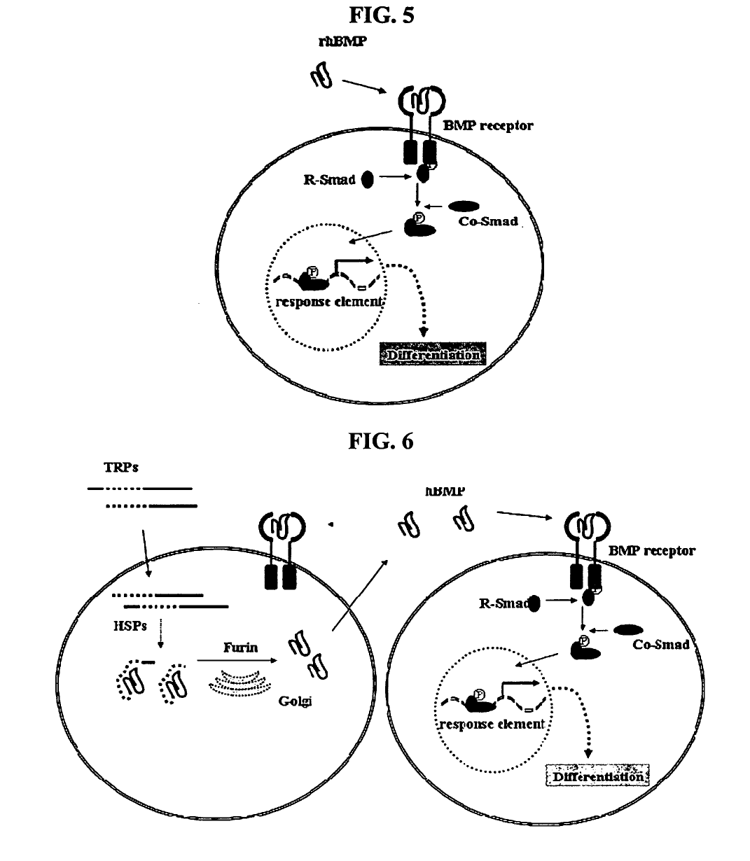

The non-activated TRPs according to the present invention have pharmacological mechanisms completely different from those of the previously known active rhBMPs, TGF-β, and the like. That is, when the non-activated TRPs according to the present invention are administered in vivo to human beings or mammals, they can stimulate the formation of bones or cartilages or improve the fibrosis or cirrhosis of kidneys, liver, lungs and heart, according to pharmacological mechanisms completely different from those of previously known rhBMPs or TGF-β proteins. For example, the prior rhBMPs, as shown in FIG. 5, bind to native receptors from cells and induce the differentiation of bone formation cells through the Smad signaling pathway, whereas the inventive TRPs, as shown in FIG. 6, permeate the cell membranes independently of the receptors and are restructured by HSP, etc., in cells, and the restructured TRPs are cleaved and activated by proprotein convertase, such as furin, which is present in golgi complexes and endosomes, and the activated BMPs are secreted out of the cells, and the secreted BMPs bind to the receptor of autocrine or paracrine cells to induce the differentiation of bone formation cells.

Also, the present invention adopts a therapeutic method completely different from the prior method attempted to induce bone formation. Namely, according to the present invention, a non-activated polypeptide (TRP) consisting of PTD, FAD and TRD is prepared in recombinant bacteria, and the prepared polypeptide is administered to human patients or mammals. The TRP is refolded in cells in vivo by HSP (heat shock protein), etc. and is cleaved by furin, etc., so as to be refolded to an activated BMP, which is then secreted extracellularly so as to induce bone formation.

The TRPs according to the present invention are polypeptides containing: PTD making the polypeptide to permeate a cell membrane without cell membrane receptors; FAD having at least one proprotein convertase cleavage site and making non-activated TRD to be activated in cells by cleavage with the proprotein convertase; and non-activated TRD which can be activated in cells by the proprotein convertase cleavage of the FAD and which can stimulate tissue growth, formation or regeneration. The inventive TRPs are inactive by themselves, but after permeation into organisms or cells, the FAD is cleaved by proprotein convertase found in most cells at large amounts so as to activate the TRD, and the activated protein is secreted extracellularly to exhibit tissue regeneration potencies. These TRPs according to the present invention can solve all the problems of the previously known active proteins that are expensive to be produced, not easy to be stored and handled.

In the present invention, a recombinant vector was first prepared by inserting the base sequence of a BMP pro-domain having a furin cleavage site and the base sequence of PTD in front of the 5′ region of a BMP gene, and inserting in front thereof a base sequence for tagging, at least four histidine-encoding base sequences for separation and purification, and ATG as an initiation base sequence.

In the present invention, as the BMP gene, a gene encoding hBMP2 set forth in SEQ ID NO: 1 or hBMP7 set forth in SEQ ID NO: 6 was used but not limited thereto. Also, as the FAD, an hBMP2 pro-domain set forth in SEQ ID NO: 14 or an hBMP7 pro-domain set forth in SEQ ID NO: 19 was used but not limited thereto. For example, the pro-domains of other BMPs and TGF-β proteins may also be used without limitations if they have a proprotein convertase cleavage site, such as furin, and can be cleaved by proprotein convertase in cells to activate TRD.

Also, as a PTD, TAT (YGRKKRRQRRR: SEQ ID NO: 27) was used in the present invention but is not limited thereto. For example, PTDs, such as drosophila melanogaster-derived Antp peptide, VP22 peptide (Gene Therapy, 8:1, Blackbirch Press, 2001) and mph-1-btm (US 2005/0147971 A1), may also be used. Also, as a tagging base sequence, an X-press tag was used, but Flag, Myc, Ha, GST, etc., may also be used.

To construct the recombinant vector, pRSET, which is resistant to ampicillin and commercially available, was used in the present invention but is not limited thereto. For example, a bacterial vector having kanamycin as selective marker, a mammalian cell expression vector, such as pcDNA, and viral vectors, such as pPGS and pBabe, may also be used.

Then, microorganisms transformed with the recombinant vector were cultured to express the inventive TRPs, and the expressed TRPs were separated and purified.

In the present invention, the transformed E. coli may be cultured in a generally used medium, and IPTG is preferably added in order to induce the over-expression of the fusion polypeptides.

In the present invention, although E. coli was used as the transformed microorganisms, it is possible to use other kinds of bacteria, yeasts or molds. Furthermore, only active sites may also be chemically synthesized and used without using microorganisms.

The fusion polypeptides expressed by the culture of the transformed microorganisms can be isolated using a GST-fusion protein or other conventional methods for protein separation and purification. For example, the TRPs according to the present invention may be purified through inducing the precipitation of the proteins using the concentration gradient of urea or ammonium sulfate and dialyzing the precipitates to remove salts. Also, because the present invention does not require the two-dimensional or three-dimensional structure of the over-expressed polypeptides, it is preferable that the proteins having a two-dimensional or three-dimensional structure are converted to one-dimensional linear structure.

When the inventive TRPs are administered into, e.g., bone cells, progenitor cells and stem cells, the FAD of the TRPs are cleaved by proprotein convertase, such as furin, to activate TRDs (e.g., BMP), which are then secreted. In other words, the TRPs permeated into cells specifically bind to F-actin in the cells, and recover a three-dimensional structure by factors, such as HSP70, and their furin cleavage sites are cleaved by furin, which is an intracellular cleavage enzyme, to activate TRDs (e.g., BMPs), which are then secreted extracellularly. The inventive TRPs introduced into cells have a half-life of 3-24 hours depending on the type and activity of the cells, indicating that the permeated proteins have activation time which varies depending on the type of cells.

Meanwhile, when the furin cleavage sites were mutated, activated BMPs were not secreted. This suggests that the protein modification by the furin cleavage enzyme is critical to the intracellular processing and activation of the permeated proteins. Also, when HBMP is naturally synthesized and processed in vivo, a signal peptide is known to play an important role in the intracellular and activation of proteins. However, even when the signal peptide was deleted in the present invention, the resulting BMP showed effects similar or better than those of wild-type BMP2 in bone formation. This suggests that the transfer of the inventive BMP using TAT and FAD does not require the signal peptide unlike the natural hBMP in vivo.

When the inventive TRPs are administered to cells at a concentration of more than 0.1 nM, the TRPs transduce into the cells in a concentration-dependent manner and converted into activated BMPs in the cells, and the activated BMPs are secreted out of the cells. The inventive TRPs directly permeated the cell membrane within one hour independently of a BMP receptor present in the cell membrane, and the cell membrane permeation process was found to be temperature-independent, indicating that the permeation process does not require the cell membrane receptor. Also, even when various concentrations of TRPs were administered to cells, cytotoxicity was not observed.

As a result, the inventive non-activated TRPs do not need to be maintained in three-dimensional structures, unlike previously known active BMPs, and they permeate bone cells in the form of a one-dimensional linear structure and are converted to active BMPs, and then are secreted out of cells and show tissue regenerative potencies. While the prior hBMPs directly show bone morphogenetic potencies, the inventive TRPs show tissue regenerative potencies through indirect activation in vivo. Thus, the inventive non-activated TRPs do not require additional equipment or cost for maintaining a three-dimensional structure, and it is very easy to separate and purify them. Also, the TRPs are produced in simple processes at a low cost, and they provide increased medical efficiency. These advantages suggest that the inventive TRPs can solve all the problems of the prior rhBMP proteins. The characteristics of the inventive TRPs are shown in Table 1 below in comparison with the prior activated rhBMP2 and rhBMP7 proteins. Table 1 shows the characteristics of processes for the separation, purification, storage and administration of the inventive TRP products in comparison with those of the previously known activated rhBMPs.

TABLE 1

| | Prior bone morphogenetic proteins | Inventive fusion polypeptides |
|---|---|---|
| Items | (rhBMP-2 or rhBMP7) | (TRPs) |
| Production method | Culture transformed CHO cells | Culture transformed |
| | | E. Coli |
| | Produce while maintaining | Maintain transformed |
| | transformed CHO cells | E. coli in a simple manner |
| | Separate from large cell medium, | Separate directly from |
| | and concentrate | E. coli, and dilute |
| Production cost | Very high | Significantly lower than |
| | | those of the prior art |
| Production equipment and | Require large-scale equipment | Very simple |
| facilities | and facilities | |
| Products | Activated rhBMP2, rhBMP7 | Fusion polypeptides of |
| | | PTD-FAD-TRD |
| Three-dimensional | Active three-dimensional structure | Non-activated peptides |
| structure | (FIG. 1 and FIG. 2) | |
| | Naturally occurring structure | Naturally non-occurring |
| | | random structure |
| Solubility in cleaning | Soluble; | Insoluble; |
| material and physiological | Loss of three-dimensional structure | No effect on structure and |
| saline | and activity | activity |
| Need of carrier for | Diffuse rapidly due to water- | Are insoluble and transducer |
| administration in vivo | solubility. Need suitable carrier | rapidly into adjacent cells. |
| | for local administration | No need carrier for local |
| | | administration |
| Stability of storage | Loss of activity upon breakdown of | Independent on structure; |
| | three-dimensional structure; Not | Storable above 37° C.; |
| | storable above 37° C.; low stability | Good stability of storage |
| | of storage | |
| Biological mechanism | Directly bind to cell membrane | Activated proteins are |
| | receptors (direct action) | secreted out of cells |
| | | (indirect action) |
| Intracellular specific | No relevant data | F-actin |
| structure binding to protein | | |
| after administration | | |
| Intracellular processing | No relevant data | Activation by cleavage with |
| | | furin enzyme |
| Temperature dependency | Needs live body temperature to be | Permeate all living cells |
| in administration | bound to receptors | independently of temperature |
| Half time for activation | No relevant data | 3-12 hours |
| Cytotoxicity | No cytotoxicity at 200 nM concentration | |
| Funtion of signal peptide | Necessary | Not necessary |
| in process for | | |
| protein activation | | |
| Cell selectivity | Signal only through | Permeable all kinds of cells |
| | BMP receptors | without help from receptors |

| | |
|---|---|
| Medical potency | Similar |
| Administration mode | Same |

The inventive non-activated TRPs shall be useful for the treatment of bone diseases. For example, the TRPs can either induce the growth of bones in an environment where bones are not normally formed, or improve bone resorption by bone fracture resulting from external injury, or stimulate the fixation of artificial joints. Specifically, the inventive TRPs shall be used for the treatment of congenital anomaly, tumor excision and reconstruction, craniomaxillofacial deformity, periodontal disease, etc., to provide an environment for inducing the differentiation of bone formation cells or stimulating the growth of bone formation cells. In addition, the TRPs according to the present invention can be used to prevent the fibrosis of tissues, such as kidneys or liver, and to induce the regeneration of tissues, and may also be used for other applications, such as the regeneration of nerves or blood vessels.

The non-activated TRPs according to the present invention can be used by themselves or in the form of pharmaceutically acceptable acid-added salts or metal complexes, for example, of zinc or iron salts. Preferable examples of the acid-added salts, which can be used in the present invention, include hydrogen chloride, hydrogen bromide, sulfate, phosphate, maleate, acetate, citrate, benzoate, succinate, malate, ascorbate, and tartarate.

The inventive composition containing the non-activated TRP as an active ingredient is preferably prepared by mixing and diluting the active ingredient with a pharmaceutically acceptable excipient or matrix carrier or by sealing the active ingredient into a receptacle-like carrier, depending on administration routes and modes and intended therapeutic uses. Moreover, the inventive composition may also be used in combination with other drugs useful for the treatment of bone defects. In this case, the preparation of a physiologically acceptable composition having the desired pH, isotonicity and stability may be performed using any conventional method known in the art to which the present invention pertains. The matrix used in the present invention may be selected depending on bioadhesion, biodegradability, mechanical properties, attractive appearance and contact properties. Examples of the carriers, which can be used in the present invention, include biodegradable and chemical substances, such as calcium sulfate, tricalcium phosphate, hydroxyapatite and polylactic acid; biodegradable and biological substances, such as bone or skin collagens, and other pure proteins or cellular matrix components; non-biodegradable and chemical substances, such as sintered hydroxyapatite, bioglass, aluminate and other ceramics; combinations of the above substances, such as polylactic acid, hydroxyapatite, collagen and tricalcium phosphate. However, the present invention is not limited to the above-mentioned carriers.

Examples of excipients, which can be used in the present invention, include lactose, dextrose, sucrose, sorbitol, mannitol, calcium silicate, cellulose, methylcellulose, microcrystalline cellulose, magnesium stearate, polyvinylpyrrolidone, water, methylhydroxybenzoate, propylhydroxybenzoate, talc, and mineral oil.

Meanwhile, the composition containing the non-activated TRP according to the present invention is preferably used in the form of an injection solution or capsule in order for the composition to be introduced into bone defect sites. The dose of the inventive composition can be determined considering the kind of excipients or matrix carriers used, the weight of formed bones, bone injury sites and the condition of injured bones in patients, the patient's age, sex and diet, disease severity, administration period, and other clinical factors. Thus, for example, a conventionally known effective amount of the composition may be administered at one time or in portions, considering the weight of bones, and additional administration can be determined while the growth of bones is observed.

### EXAMPLES

Hereinafter, the present invention will be described in more detail by examples. However, it will be obvious to those skilled in the art that the present invention is not limited by these examples, and various variations and modifications to theses examples are possible without deviating from the sprit and scope of the present invention.

Particularly, although the following examples illustrated only hBMP2 and hBMP7 as TRDs contained in TRPs, those skilled in the art will appreciate that it is possible to use not only human BMPs, such as hBMP3, hBMP4, hBMP6, and hBMP14 (MP-52), but also BMPs derived from mammals, such as rats, cattle, pigs, and other animals. Furthermore, those skilled in the art will appreciate that it is possible to use, in addition to BMPs, other kinds of polypeptides, such as a group of TNF-αproteins, including TGF-β, β-NGF (β-nerve growth factor), β-amyloid, ADAMs (a disintergrin and metalloproteinase-like), and ectodysplasin-A (Eda-1), a group of MMPs, including MT1-MMP (membrane type-matrix metalloproteinase) and MMP-2, and insulin-like growth factor-1 (IGF-1).

Moreover, although the following examples illustrated only the pro-domains of BMPs, as FADs contained in TRPs, those skilled in the art will appreciate that any pro-domain may be used without limitations if it has a proprotein convertase cleavage site and is cleaved by proprotein convertage in cells to activate TRDs.

Example 1: Preparation of ITAT-hBMP2] fusion polypeptide

Each of proteins belonging to a BMP/TGF-β group consists of a dimer comprising the same two peptides linked to each other by one disulfide bond. In this regard, each of the peptides consists of 120-140 amino acids depending on the type of BMPs, one of 7 cystein terminal groups present in BMPs forms a disulfide bond with the same site of the other peptides to form a dimer, and the remaining 6 cysteins form 3 intrachain disulfide bonds in the same amino acids, resulting in a unique three-dimensional structure (Proc. Natl. Acad. Sci., 93:878, 1996; J Bone Joint Surg., 83:S1, 2001). Herein, BMP2 consists of 114 amino acids, BMP7 consists of 139 amino acids, and each of TGF-β and β3 consists of 112 amino acids. FIG. 1 shows the amino acid sequences of these peptides and a schematic diagram of the three-dimensional structure thereof, and FIG. 2 shows the three-dimensional structure of previously known BMP2 (Eur. J Biochem., 237:295, 1996; J Mol. Biol., 287:103, 1999). The locations of the seven cysteins in BMP/TGF-β are all conserved, suggesting that the cysteins play an important role in the three-dimensional structure.

Accordingly, as well known in the art, when a fusion protein prepared by binding PTD to the previously known hBMP2 is introduced into cells, it is expected that this fusion protein will permeate the cell membrane even without the cell membrane receptor so that it will be refolded into a biologically active protein by, e.g., HSPs (heat shock proteins) (Nature Med., 4:1449, 1998; Science, 285:1569, 1999).

For this reason, in this Example, a gene encoding hBMP2 having an amino acid sequence of SEQ ID NO: 1 was amplified by RT-PCR using the total mRNA of Saos-2 cells (American Type Culture Collection, ATCC HTB-85) as a template with primers of SEQ ID NOs: 28 and 29. For cloning into a bacterial expression vector, a restriction enzyme Kpn 1 site (5′-ggtacc-3′) was added to the 5′ region of each of the primers. Cloning primers used in Examples below also contained the restriction enzyme Kpn I site.

| | |
|---|---|
| SEQ ID NO: 28: | |
| 5′-caa gcc aaa cac aaa cag cgg aaa-3′ | |
| SEQ ID NO: 29: | |
| 5′-ttt gct gta cta gcg aca ccc | |
| | aca-3′ |

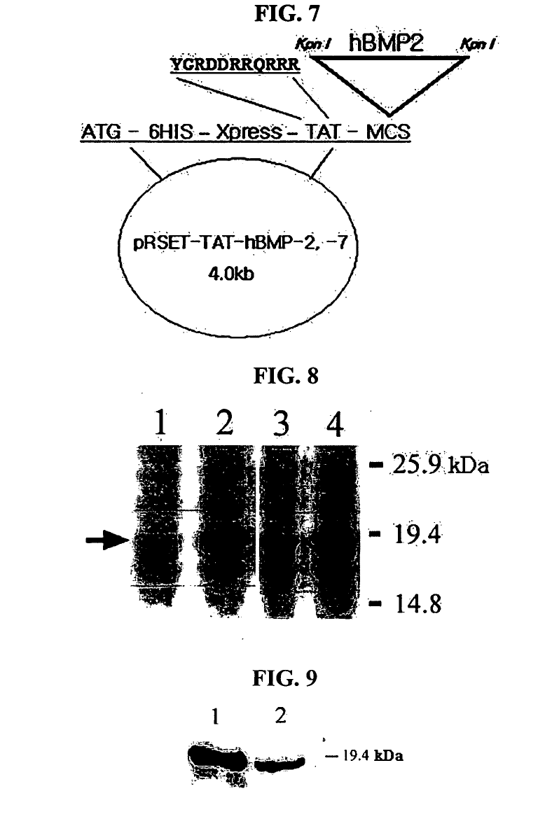

The amplified hBMP2 gene was inserted into a TA cloning vector (Invitrogen, Inc). It was confirmed that the 114 amino acids are contained by comparison with a base sequence (NCBI, NM_001200) in GenBank. The cloned hBMP2 gene was subcloned again into the KpnI sites of a pRSET bacterial expression vector (Invitrogen, Inc), the base sequence of TAT (YGRKKRRQRRR: SEQ ID NO: 27) was inserted in the 5′ region of the hBMP2 gene. Then, in front thereof, an X-press (Invitrogen, Inc) tag, six histidine-encoding base sequences for separation and purification, and initiation base sequence ATG, were inserted, thus constructing a recombinant expression vector for the expression of BMP2 (see FIG. 7). As a negative control, a vector inserted with no TAT was used.

The constructed recombinant vector was introduced into E. coli BL21 (Invitrogen Inc.) by a conventional heat shock method, and cultured at 37° C for 2-3 hours. To the culture medium, 1 mM of IPTG (isopropylthio-galactoside) was added, and the resulting culture medium was additionally cultured for 2-18 hours to induce the expression of TAT-BMP2.

The culture broth was centrifuged and then the cell pellet was collected. 8M of a urea solution was added into the cell pellet to remove the two-dimensional and three-dimensional structures of BMP. Ni-Ti beads (Qiagen) were added thereto so as to bind the TAT-BMP2 to the beads, after which the beads were washed three times with the same solution and eluted using imidazole and high-salt buffer, thus obtaining a purified TAT-BMP2.

FIG. 8 shows the results of electrophoresis for the total protein before and after inducing protein expression by IPTG during the culture of the transformed E. coli BL21. In FIG. 8, the arrow represents the TAT-BMP fusion protein induced by IPTG, lane 1 represents uninduced TAT-BMP2, lane 2 represents TAT-BMP2 induced by IPTG, lane 3 represents uninduced BMP2 (w/o TAT), and lane 4 represents BMP2 (w/o TAT) induced by IPTG. As shown in FIG. 8, in the case where the production of the fusion proteins was induced by the addition of IPTG, the TAT-BMP2 fusion protein was produced in a large amount compared to the control group.

FIG. 9 shows the results of Western blot analysis using an anti-X-press antibody for the purified TAT-BMP2 and BMP2 (w/o TAT). In FIG. 9, lanes 1 and 2 represent TAT-BMP2 and BMP2 (w/o TAT), respectively. As shown in FIG. 9, the addition of TAT (˜20kDa) resulted in a slight increase in molecular weight compared to the case of no TAT addition (˜18kDa). These results suggest that the TAT-hBMP2 fusion polypeptide was successfully prepared.

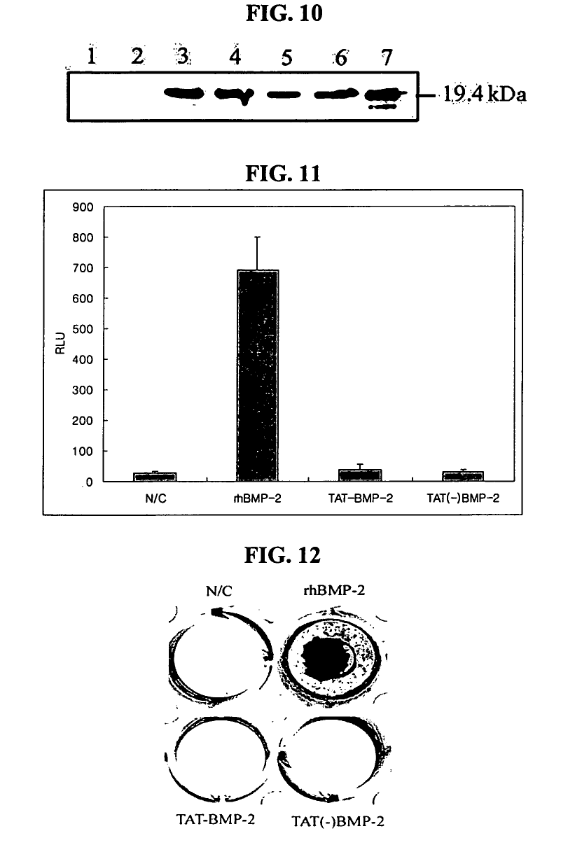

In order to examine whether the prepared TAT-BMP2 permeates the cell membrane, primarily cultured gingival fibroblasts were treated with the TAT-BMP2 at a concentration of 4 nM for 2 hours, and the cells were collected and Western-blotted with an anti-X-press primary antibody (Invitrogen, Inc). As a result, as shown in FIG. 10, the TAT-BMP2 mostly permeated the cell membrane within 2 hours, and the peptide having no TAT did not permeate the cell membrane. Lane 1 shows the result of Western blot for 4 nM of the TAT-free BMP2 treated for 2 hours, and lanes 2 through 6 show the results of Western blot using an anti-X-press antibody for the intracellular expression of the same concentration of TAT-BMP2 proteins treated for 0 hr, 15 min, 30 min, 1 hr and 2 hr, respectively. Lane 7 is a Western blot positive control analyzed using 10 ng of TAT-BMP2.

In order to examine whether TAT-BMP2 introduced into cells is refolded to biologically active BMP2 proteins, which then induce the differentiation of bone cells, 2 nM of TAT-BMP2 was administered to the same cells at 48-hr intervals, and after one week, the cells were measured for alkaline phosphatase (ALP) activity. After culturing for 2 weeks, the cells were subjected to von Kossa staining to observe the formation of mineralized substances. As a negative control, TAT-free BMP2 was used, and as a positive control, commercially available rhBMP-2 was used. FIG. 11 shows ALP activities. In FIG. 11, the negative control (N/C) is a result for primarily cultured fibroblasts treated only with a medium for one week, rhBMP-2 is a result for cells treated with commercially available BMP2 at a concentration of 2 nM for one week, TAT-BMP-2 is a result for cells treated with TAT-BMP-2 of this Example at a concentration of 2 nM for one week, and TAT(-)BMP-2 is a result for cells having TAT deleted from TAT-BMP-2. FIG. 12 shows the results of von Kossa staining conducted to observe the deposition of mineralized substances. As shown in FIG. 11 and FIG. 12, the TAT-hBMP-2 of this Example had no biological activity, unlike commercially available rhBMP-2.

Example 2: Preparation of ITAT-FAD-hBMP21 fusion polypeptide (TRP-1)

As described in Example 1, hBMP2 shows biological activity by forming a characteristic internal three-dimensional structure and a dimer by a disulfide bond. However, as described in Example 1, a fusion protein of TAT and hBMP2 had no biological activity to induce bone cell differentiation even though it successfully permeated the cell membrane. This means that proteins having biological activity by secretion are insufficient to show activity only with the restructuring of amino acids by, e.g., HSPs, unlike the prior PTD-fused proteins (Trends Cell BioL, 10:290, 2000; Curr. Prot. Pept. Sci., 4:97, 2003).

BMPs and TGF-β are present in a form of precursor consisting of more than 400 amino acids, when biosynthesized in intracellular ribosomes. The synthesized amino acids transport to Golgi complexes, endosomes, etc., by signal peptides present in the N-terminal region, and cleaved and activated by proprotein convertase, such as furin, and then secreted extracellularly (Nature Rev. Mol. Cell Biol., 3:753, 2002; J Cell Biol., 144:139, 1999). The proteins are subjected to post-translational modification in the golgi complexes, etc., in which a signal peptide and prodomain located at the amino N-terminal region play a critical role (Mol. Biol. Cell, 15:5012, 2004). In this process, the BMP precursor is activated into a mature BMP moiety by the cleavage of the furin cleavage site (-RSKR-) with furin and then secreted, similarly to other secretory proteins (Constam, D.B. & Robertson, E.J., J. Cell Biol., 144:139, 1999; Cui, Y. et al., Genes & Development, 15:2797, 2001). However, in said article, there is neither mention of a method for the intracellular administration of the BMP precursor, nor suggestion that the BMP precursor is introduced into cells in the form of a fusion with, particularly PTD.

Those known proprotein convertases, such as furin, include PC7, PC5/6A, PC5/6B, PACE4, PC1/3, PC2, and PC4 (Nature Rev. Mol. Cell Biol., 3:753, 2002). They are abundantly found in most of intracellular organelles, particularly Golgi networks, endosomes, and secretory granules, and play important roles not only in the activation of various proteins, but also in the activation of infectious diseases. Human mature proteins, which are activated by proprotein convertase, such as furin, include, in addition to BMPs/TGF-β a group of TNF-α proteins, such as β-NGF (β-nerve growth factor), β-amyloid, ADAMs (a disintergrin and metalloproteinase-like) and ectodysplasin-A(Eda-1), a group of MMPs, including MT1-MMP (membrane type-matrix metalloproteinase) and MMP-2, and IGF-1 (Nature Rev. Mol. Cell Biol., 3:753, 2002).

It is particularly known that, when BMPs are naturally biosynthesized in cells, one or two furin cleavage sites in the prodomain of the BMP precursor contribute to the activation of BMPs (J. Cell BioL, 144:139, 1999; Genes Dev., 15:2797, 2001). In the case of human BMP2, one or two furin cleavage sites (RXKR, wherein R: Arg, K: Lys, X: other amino acids except for basic amino acids) are present in the prodomain and were deduced to play an important role in the activation and secretion of the PTD-BMP2 fusion protein. To prove this hypothesis, in this Example, a polypeptide having a structure of TAT-FAD-hBMP2 was prepared by adding a furin cleavage site-containing FAD having an amino acid sequence of SEQ ID NO: 14 to the TAT-hBMP2 of Example 1.

For this purpose, to add the FAD-encoding base sequence, DNA having both a base sequence encoding hBMP2 of SEQ ID NO: 1 and a base sequence encoding FAD having an amino acid sequence of SEQ ID NO: 14 was amplified in the same manner as in Example 1 using primers of SEQ ID NOs: 30 and 31. The base sequence of the amplified DNA was confirmed and then, the gene was cloned into a bacterial expression vector in the same manner as in Example 1.

SEQ ID NO: 30:
5′-gag ttt ttc cat gtg gac gct ctt-3′
SEQ ID NO: 31:
5′-ttt gct gta cta gcg aca ccc aca-3′

The transformed E. coil was cultured and purified in the same manner as in Example 1, thus obtaining TAT-FAD-hBMP2 (TRP-1). Namely, the culture broth having TRP-1 produced in E. coli was centrifuged, and to the supernatant and cell pellet, 8M of urea solution was added to remove the two-dimensional and three-dimensional structures of BMP2. To the remaining substance, Ni-Ti beads (Qiagen) were added so as to bind TRP-1 to the beads, and the beads were washed three times with the same solution and eluted using imidazole and high-salt buffer, thus obtaining a purified TRP-1 (see FIG. 3).

Meanwhile, to confirm the importance of the signal peptide, the prodomain and the furin cleavage site, the constructed expression vector as a template and each of primers of SEQ ID NOs: 32 and 33 (ΔSig), SEQ ID NOs: 34 and 35 (ΔPro), and SEQ ID NOs: 36 and 37 (R283A), were used to construct the respective deletion or mutation vectors. A schematic diagram of these vectors is shown in FIG. 13.

SEQ ID NO: 32:
5′-tcc acc atg gcc ggt acc ctc gtt ccg gag ctg
ggc-3′
SEQ ID NO: 33:
5′-gcc cag ctc cgg aac gag ggt acc ggc cat ggt
gga-3′
SEQ ID NO: 34:
5′-tcc acc atg gcc ggt acc gat gga aaa ggg cat
cct-3′
SEQ ID NO: 35:
5′-agg atg ccc ttt tcc atc ggt acc ggc cat ggt
gga-3′
SEQ ID NO: 36:
5′-ctc cac aaa aga gaa aaa gct caa gcc aaa cac
aaa cag-3′
SEQ ID NO: 37:
5′-ctg ttt gtg ttt ggc ttg agc ttt ttc tct ttt
gtg gag-3′

E. coli was transformed with each of the constructed expression vectors and then cultured and purified in the same manner as described for TRP-1, thus obtaining several TRP-1 variants. In FIG. 13, TRP-1 represents TAT-FAD-hBMP2 according to this Example, which comprises FAD added to TAT-hBMP2; Δ(Sig), was obtained by deleting a signal peptide portion (amino acid residues 1-24 of SEQ ID NO: 14) from TAT-FAD-hBMP2; Δ(FAD) was obtained by deleting an N-terminal FAD (amino acid residues 1-269 of SEQ ID NO: 14) from TAT-FAD-hBMP2 such that the Δ(FAD) had only TAT-hBMP2 and a second furin cleavage site; and R283A was obtained by mutating the Arg of the second furin cleavage site of TAT-FAD-hBMP2 to Ala. The N-terminal region of each of the expression vectors contained a TAT domain, a X-press tag, etc., as in the case of Example 1, and after construction, the base sequence of each of the vectors was analyzed.

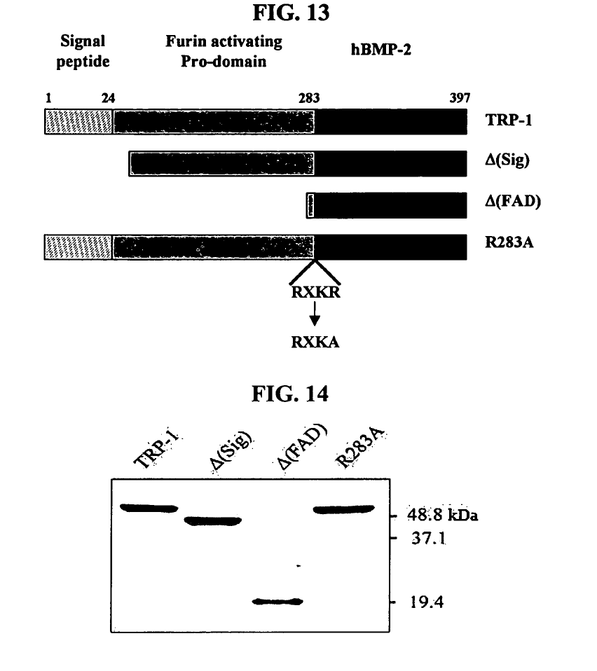

Next, each of the above-prepared TRP-1 and its variants was introduced into the same kind of cells. Namely, primarily cultured fibroblasts were treated with each of the recombinant proteins at a concentration of 2 nM for 2 hours, and the cells were collected and Western-blotted with an anti-X-press antibody (FIG. 14). As a result, as shown in FIG. 14, TRP-1 and its variants were all permeated into the cells and detected.

Example 3: Cleavage and activation of TRP-1 bv proprotein convertase

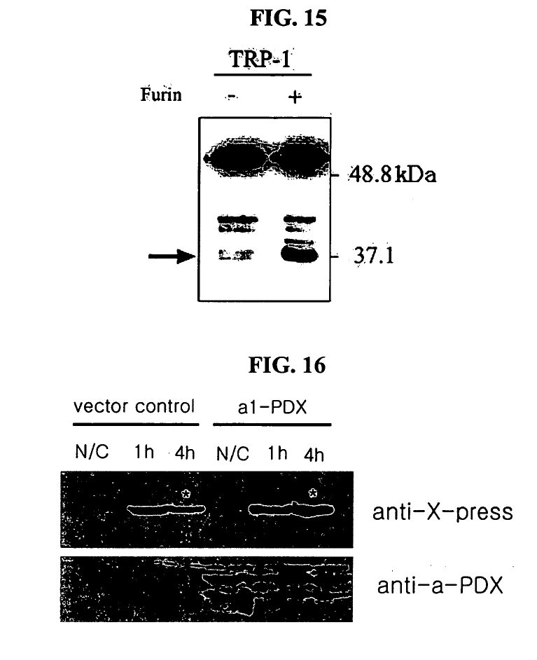

In order to examine whether the inventive non-activated TRP-1 is cleaved and activated by furin in cells, TRP- 1 prepared in Example 2 was cleaved in vitro using a recombinant furin protein (Sigma, USA). As a result, as shown in FIG. 15, TRP-1 was successfully cleaved in vitro by the furin.

Also, in order to examine whether the inventive non-activated TRP- 1 is activated by furin in cells, intracellular furin was inhibited using aL antitrypsin Portland (α1-PDX) expression vector (Proc. Natl. Acad. Sci., 95:7293, 1998), as a furin inhibitory protein. Because the inventive non-activated TRP-1 is converted to a biochemically active protein after introduction into cells, the amount of TRP- 1 initially introduced into cells shall gradually decrease at a given time after the administration of TRP-1. Thus, it can be expected that, when furin is inhibited by inducing the expression of α1-PDX, the remaining amount of TRP shall increase.

As a result, as shown in FIG. 16, the half-life of the inventive TRP- 1 was increased by the expression of α1-PDX (i.e., the inhibition of furin enzyme). Namely, when administered with the inventive TRP-1, the group administered with α1-PDX showed an increase in half life compared to the control group after 4 hours (asterisked). This result suggests that TRP- 1 administered into cells is cleaved and activated by furin enzyme.

Example 4: Importance of FAD in activation of TRP-1

It was confirmed through Examples 2 and 3 that TRP-1 is cleaved and activated by furin in cells. Thus, it can be expected that FAD and the furin cleavage site will play an important role in making the TRP-1 to show biological activity after administration into cells.

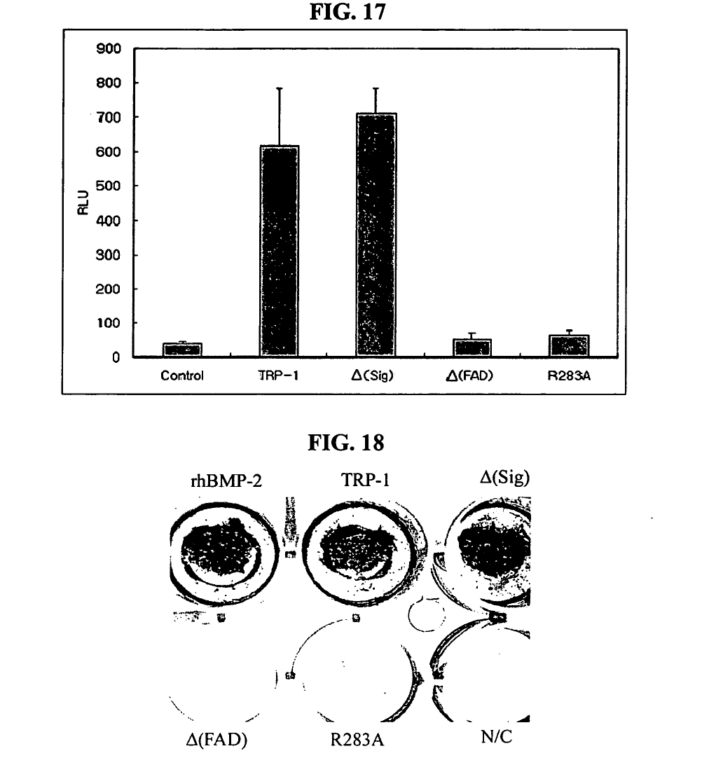

To confirm this expectation, TRP-1 obtained in Example 2 and its variants were added to primarily cultured fibroblasts and observed for ALP activity and the deposition of mineralized substances (FIG. 17). TRP-1 and its variants were measured for alkaline phosphatase (ALP) activity in the same manner as in Example 1, as a result, as shown in FIG. 17, TRP-1 (TAT-FAD-hBMP2) showed high ALP activity, and the TRP-1 variant [Δ(Sig)] from which a signal peptide has been deleted showed a similar or higher activity than that of TRP- 1. These results indicate that the signal peptide is not necessary for the activation of BMP2 by TAT and FAD. However, it can be seen that the variant [Δ(FAD)] containing only a second furin activation site in TAT-hBMP2, and the variant (R283A) having a mutation in a portion of the furin cleavage site of TRP-1, had no biological activity.

Also, each of TRP-1 and its variants were treated for 2 weeks according to the above-described method and stained by the von Kossa method and then observed for the formation of mineralized substances. As a result, as shown in FIG. 18, the mineralization inducing effects were similar to ALP activity. Herein, commercially available rhBMP-2 treated at the same concentration was used as a positive control, and an untreated medium was used as a negative control (N/C). These results suggest that, in order for the TAT-FAD-hBMP2 recombinant protein to permeate into cells and to have biological activity, a process for migration from ribosomes to golgi networks is unnecessary unlike the case of naturally biosynthesized hBMP-2 or rhBMP-2, but the furin cleavage site must be present in a complete and precise manner.

The results of this Example indicate that, when the inventive TAT-FAD-hBMP2 fusion polypeptide has a deletion of any one domain in a polypeptide consisting of 3 domains of PTD, FAD and TRD (hBMP2 in this Example) or has a defect, it cannot exhibit the desired pharmacological effects upon administration into the human body. Although this interpretation of mechanisms for the pharmacological action of TRP-1 (FIG. 6) cannot be complete, it is expected that, when polypeptides, such as non-activated BMPs or TGF-β proteins, are administered into the human body for the purpose for regenerating bones, cartilages or other various tissues, the inventive TRP-1 shall show highly useful pharmacological effects and remarkably high commercial values, compared to previously known products. Namely, the TAT-FAD-hBMP-2 was designed under the concept of new pharmacological actions completely different from previously known BMPs or TGF-β proteins and prepared according to a new method, and was thus named “TRP- 1”.

Example 5: Preparation of TRP-2[TAT-FAD-BMP7]

As described in Example 4, when secretory proteins, such as BMPs and TGF-β, are fused with PTD, it is insufficient to only make them to have full length, and upon biosynthesis in vivo, they must sufficiently have furin cleavage and activation sites in a precursor formed. Human BMP7 is known to stimulate the differentiation of bones and cartilages, and recently known to act as an antagoist of TGF-β which is a member of the same gene group, thus inhibiting the fibrosis and cirrhosis of kindenys, liver, lungs and heart (Nature Med., 9:964, 2003; J. Clin. Invest., 112:1776, 2003).

In this Example, a base sequence encoding an amino acid sequence comprising FAD of SEQ ID NO: 19 and hBMP7 of SEQ ID NO: 6 was amplified in the same manner as in Example 2 using primers of SEQ ID Nos: 38 and 39. The amplified base sequence was analyzed in GenBank (NCBI, NM_001719), and cloned into a bacterial expression vector in the same manner as in Example 2 so as to prepare a recombinant polypeptide that was then named “TRP-2”.

SEQ ID NO: 38:
5′-ggc gcg atg cac gtg cgc tc ctg-3′
SEQ ID NO: 39:
5′-agg gtc tga att ctc gga gga gct-3′

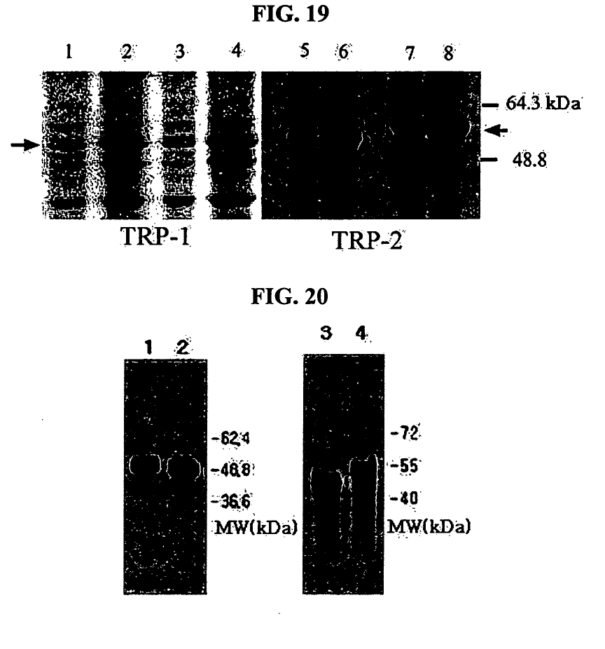

FIG. 19 shows the results of electrophoresis for a total protein before and after inducing the expression of the protein by IPTG in the culture of transformed E. coli BL21. In FIG. 19, the arrow represents a TRP fusion protein induced by IPTG; lane 1 represents uninduced TRP-1; lane 2 represents TRP- 1 induced by IPTG; lane 3 represents uninduced TRP-1 (w/o TAT); lane 4 represents TRP-1 (w/o TAT) induced by IPTG; lane 5 represents uninduced TRP-2 (w/o TAT); lane 6 represents induced TRP-2 (w/o TAT); lane 7 represents uninduced TRP-2; and lane 8 represents TRP-2 induced by IPTG. As shown in FIG. 19, in the case where the production of fusion proteins by the addition of IPTG was induced, TRPs were induced in larger amounts than that in the control group.

FIG. 20 shows the results of Western blot analysis using an anti-X-press antibody for the purified TRPs. In FIG. 20, lanes 1 through 4 represent TRP-1, TRP- 1 (w/o TAT), TRP-2 (w/o TAT) and TRP-2, respectively. As shown in FIG. 20, the addition of TAT showed a slight increase in molecular weight compared to the case of no addition, suggesting that the TRP fusion polypeptides were successfully prepared.

Example 6: Intracellular transduction of TRP-1 and TRP-2

In this Example, primarily cultured gingival fibroblasts were first treated with 4 nM of each of TRP-1 and TRP-2 purified in Examples 2 and 5, and the TRP-1 and TRP-2 present in each of the cells and media were examined for intracellular transduction by comparing their expression levels.

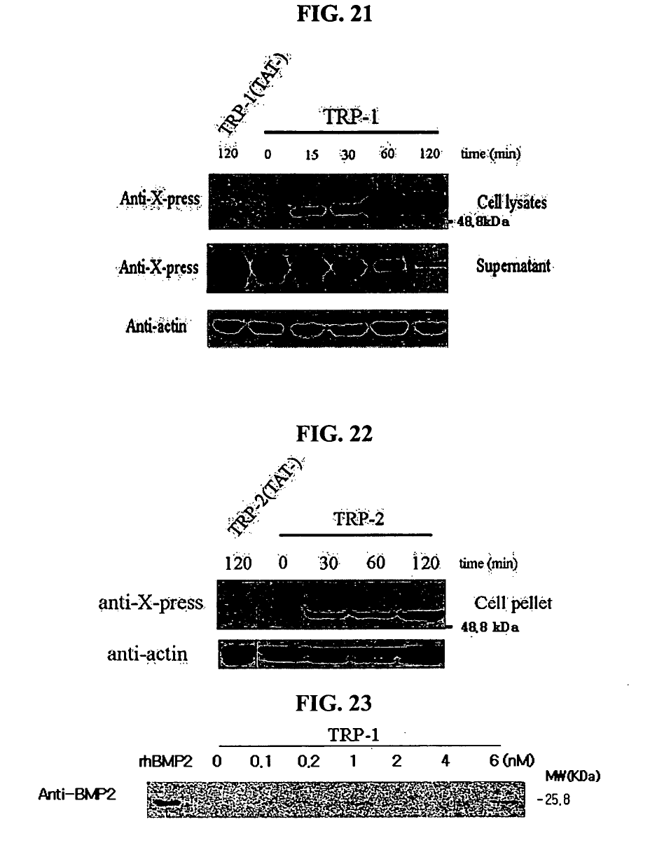

As a result, as shown in FIG. 21, TRP- 1 permeated the cell membrane and transduced into cells. Namely, the primarily cultured gingival fibroblasts were first treated with 4 nM of TRP-1, and the expression of TRP-1 present in each of the cell and medium was analyzed by Western blot. As a result, The TRP-1 proteins mostly transduced into the cells within one hour and little remained in the medium after 6 hours. However, the control group having no TAT did not enter the cells at all. Also, TRP-2 was administered into the cells in the same manner. As a result, as shown in FIG. 22, TRP-2 also permeated the cell membrane and transduced into the cells within 2 hours, but the control group having no TAT did not enter the cells at all.

Example 7: Secretion of activated BMP by TRPs and importance of PTD

In this Example, in order to examine whether the activated BMP is secreted in cells introduced with TRPs, TRP-1 was introduced into the cells and measured for the secretion of the activated BMP2 within 48 hours. As a result, as shown in FIG. 23, the TRP- 1 introduced into the cells secreted the activated BMP2 within 48 hours. Herein, rhBMP-2, which is a commercially available active BMP2, was used as a positive control.

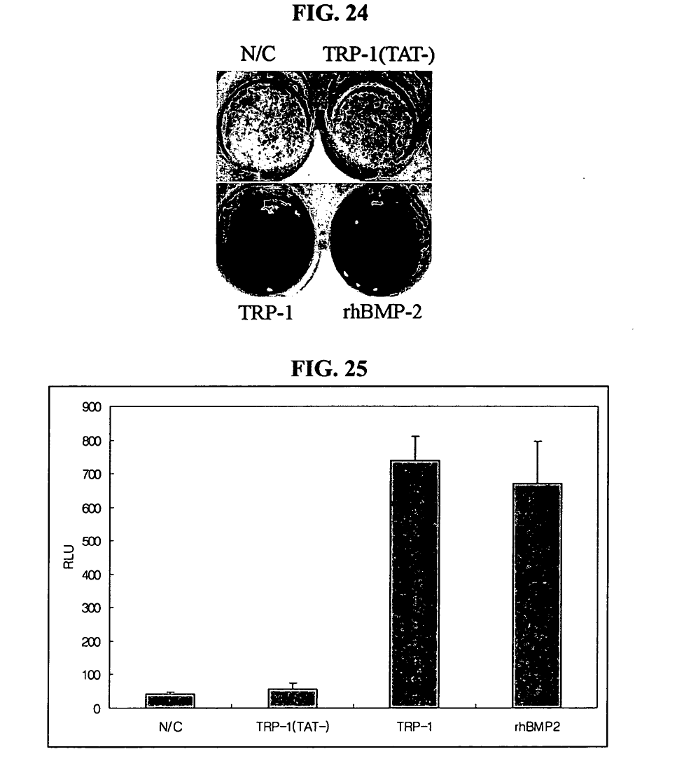

Also, the inventive TRP- 1 was introduced into the primarily cultured fibroblasts and analyzed for the differentiation of bone cells and the formation of mineralized substances, thus examining effects on the differentiation of bone cells and the regeneration of bones. For this purpose, fibroblasts were cultured for one week, being treated with each of 2 nM of rhBMP-2 and TRP- 1 for 2 weeks. After one week, the cells were subjected to von Kossa staining and observed visually, as a result, as shown in FIG. 24, the differentiation of bone cells and the formation of mineralized substances could not be observed in the case of a negative control (N/C) consisting of only the cell culture medium, and TRP-1 having no TAT (i.e., [TRP-1(TAT-)]. However, in the case of TRP-1 and commercially available rhBMP-2, the differentiation of bone cells and the formation of mineralized substances could be observed in the fibroblasts.

In addition, fibroblasts were treated in the same manner for one week and measured for alkaline phosphatase (ALP) activity used as a bone cell differentiation marker. As a result, as shown in FIG. 25, the inventive TRP-1 and the commercially available rhBMP-2 showed similar ALP induction effects.

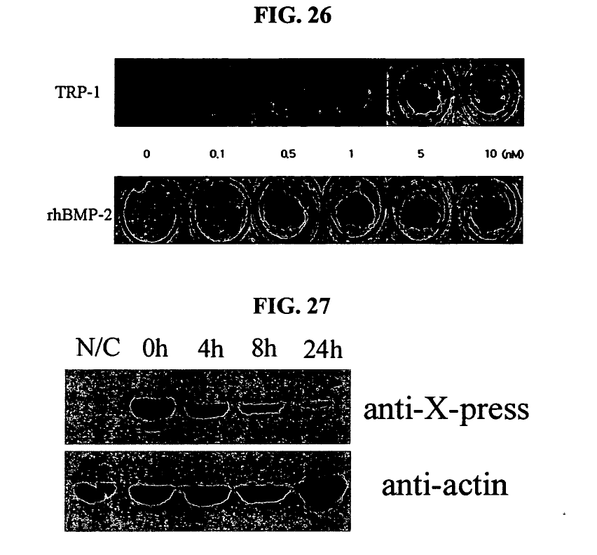

Meanwhile, in order to compare the potency of the commercially available BMP2 (rhBMP-2) with that of the inventive TRP-1, fibroblasts were cultured for one week, treated with each of TRP-1 and rhBMP-2 and subjected to von Kossa staining, followed by visual observation. As a result, as shown in FIG. 26, the inventive TRP-1 and the activated rhBMP-2 showed similar potencies. Specifically, at concentrations of higher than 0.5 nM, the commercially available rhBMP-2 and the inventive TRP- 1 showed similar potencies.

Example 8: Activation half life of TRPs after cell membrane permeation

In this Example, in order to confirm that TRPs introduced into cells are secreted in the form of activated BMPs after processes for protein rearrangement by HSPs, etc., and activation by furin, the activation half life of the administered proteins was examined. For this purpose, the 293 cell line (ATCC CRL-1573) having high furin activity was administered with the inventive TRP-1 and treated in the same manner as in Example 6. Then, the expression of the TRP-1 in the cells was analyzed hourly by Western blot.

As a result, as shown in FIG. 27, the TRP-1 administered into the cells had a half-life of 3-4 hours in the 293 cells. The half-life varied depending on the type of cells, in which the half-life was about 6 hours in the osteogenic sarcoma cell line, and about 8 hours in the primarily cultured fibroblasts. These results suggest that the inventive TRPs are secreted in the form of activated BMPs after activation by HSPs, furin, etc., in cells.

Example 9: Cell membrane permeation of TRPs and effect of temperature on permeation

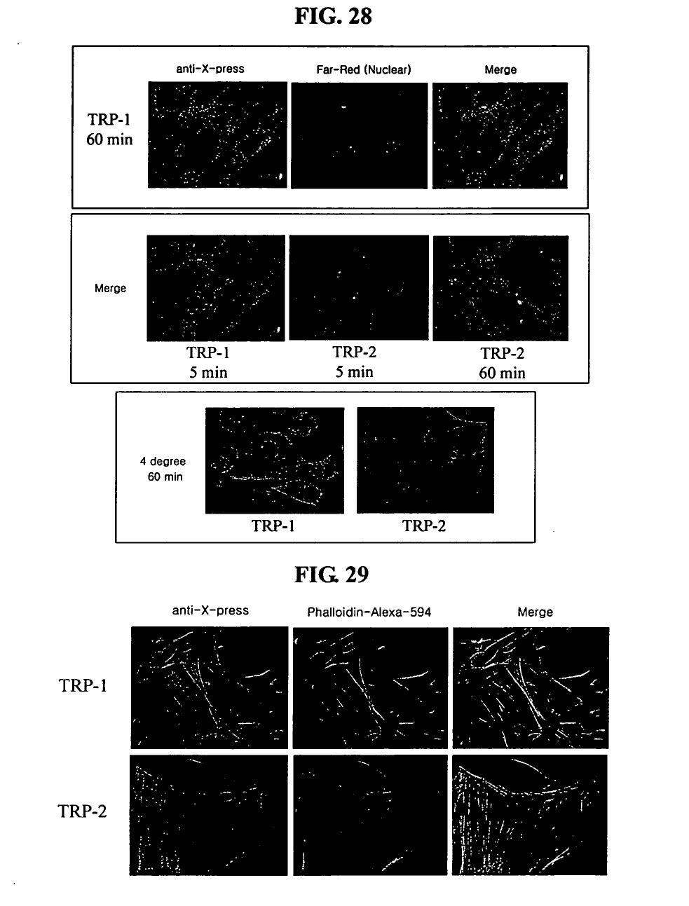

The commercially available recombinant rhBMPs pass through receptors present in the cell membrane, but the inventive TRPs directly permeate the cell membrane, and the intracellular permeation of the prior rhBMP-2 through the receptor is dependent on temperature. In order to examine whether the intracellular permeation of the inventive TRPs is dependent on temperature, the inventive TRP- 1 and TRP-2 were administered at varying temperatures, and observed with confocal fluorescent microscope at various points in time. Herein, each of the inventive TRP-1 and TRP-2 was used at a constant concentration of 10 nM and treated at various temperatures and time points as shown in FIG. 28. The permeated TRPs were allowed to react with anti-X-press (Invitrogen) as a primary antibody and Alexa-Fluoro-488 (Invitrogen) as a secondary antibody, and their nuclei were stained with Far-Red TOTO-3 (Molecular Probes). The uppermost photographs in FIG. 28 show that TRP-1 entered the cytoplasm at 37° C, and the photograph indicated by “Merge” shows the merged image of the cell nuclei with TRP- 1 having the same cross section. Also, the middle photographs show the merged images of TRP- 1 treated for 5 min and TRP-2 treated for 5 and 60 min. The lowest photographs show the merged images of TRP- 1 and TRP-2 that have been treated at 4° C for 60 min and transduced into cells.

As a result, as shown in FIG. 28, TRP-1 permeated the cell membrane within 5 minutes after administration at 37° C. After about one hour, TRP-1 and TRP-2 were mostly found in the cytoplasm although they were also partially present in the nuclei. In addition, they also showed permeation patterns at 25° C and 4° C in a manner similar to the case of 37° C. These results revealed that that the cell membrane permeation of the inventive TRPs is independent on temperature.

Example 10: Specific binding between TRPs introduced into cells and F-actin

As described above, the inventive TRPs were present in cells in the form of a linear pattern. In this Example, in order to examine whether the inventive TRPs are present specifically in a specific organelle or structure after cell membrane permeation, the TRPs were subjected to immunofluorescent staining using phalloidin specific to a F-actin stress fiber having an arrangement similar to their structure (Science, 276:1425, 1997) and observed with a confocal fluorescent microscope (FIG. 29). In this case, TRP- 1 and TRP-2 were fluorescence-stained in the same manner as in Example 9, and Phalloidin-Alexa-594 (Molecular Probes) was used for F-actin-specific staining. In FIG. 29, weak blue images in the Merge photographs show the lower portions of cell nuclei.

As a result, as shown in FIG. 29, the inventive TRP-1 and TRP-2, after administration, not only have the same arrangement as that of cellular F-actin, but also are present in the same location as that of F-actin. This indicates that TRPs specifically react and bind with F-actin after permeation into cells. These results revealed that TRPs are not randomly distributed in the cells, but are rather bound specifically to F-actin and are converted to active BMPs through various processes for activation and furin cleavage, when permeated into cells.

Example 11: Induction of bone formation by TRPs in animal test models

Examples 1 through 10 revealed that the inventive TRP-1 and TRP-2 permeate the cell membrane and are activated by furin, and the activated proteins induce the differentiation of bone cells and the formation of mineralized substances. In this Example, in order to examine whether the inventive TRPs induce bone formation in vivo, a naturally incurable defect (8 mm critical defect) was made in the skull of each of rats. Then, the inventive TRP-1 and the commercially available rhBMP-2 were administered into the defects and observed for the induction of bone formation (FIG. 30). In making the naturally incurable damage in the skull of each rat, 8-mm-diameter trephine was used to the defect. In a negative control, type I collagen was used, and in a positive control (rhBMP), a mixture of commercially available recombinant BMP2 and type I collagen was used. The inventive TRP-1 was administered in the same manner as the positive control. Also, each of rhBMP-2 and TRP-1 was administered in an amount of 10 nmole. Two weeks after the administration of BMPs, the tissue of each test group was taken and fixed in 10% formalin for 24 hours, and 4-μm sections of the tissue were stained with hematoxylin/eosin and then observed with an optical microscope. In FIG. 30, both the arrows indicate the ends of each bone defect, and the lowest box is an enlarged view of a bone formation site induced by TRP-1.

As a result, as shown in FIG. 30, the differentiation of bone cells and the formation of bones were actively induced in vivo only 2 weeks after administration with the inventive TRP-1. Also, the administration of TRP-1 showed potency better or at least similar to the commercially available rhBMP-2 administered at the same amount.

Example 12: Cytoxicitv of TRPs

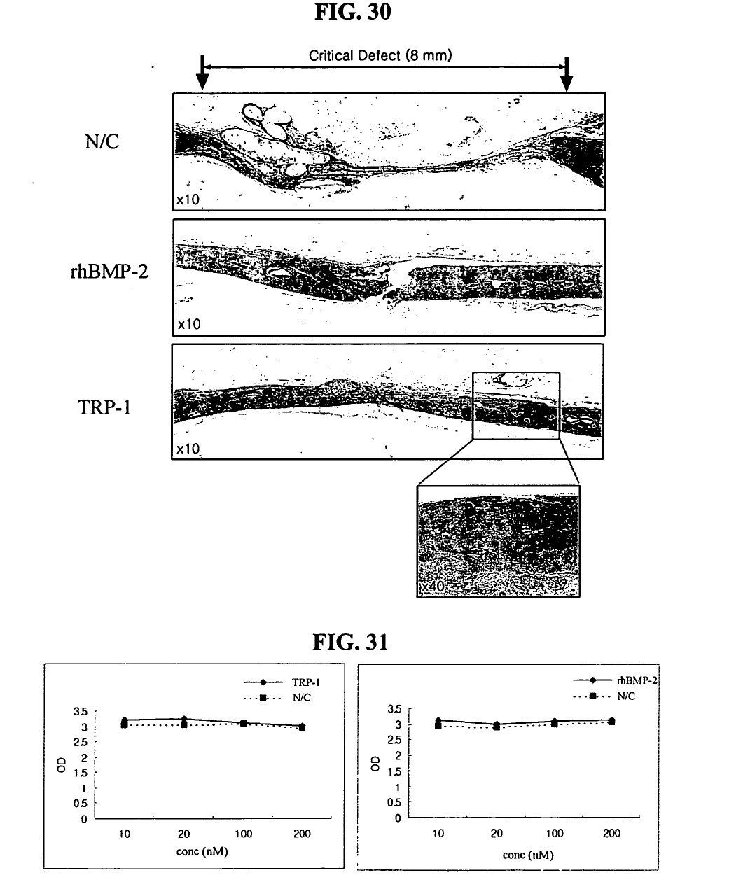

Examples 1 through 11 presented the mechanisms and potential effects of the inventive TRPs, which result from their administration to cells or tissues. In order to examine cytotoxicity occurring when the inventive TRPs were administered in excess, the inventive TRPs together with the commercially available rhBMP were analyzed for cytotoxicity while increasing their concentration to 200 nM. For this purpose, TRP-1 and recombinant BMP2 were administered to primarily cultured fibroblasts at various concentrations, and 72 hours after the administration, the number of viable cells was counted (FIG. 31). As a result, as shown in FIG. 3 1, TRP-1 did not showed any cytotoxicity even when it was administered at a concentration of 200 nM.

Although specific embodiment of the present invention was described in detail above, those skilled in the art would appreciate that these descriptions are only intended to give preferred embodiments and are not construed to limit the scope of the present invention. For example, the administration of the inventive TRP-1 to the human body will be useful to stimulate the formation of bones and cartilages, while the administration of the inventive TRP-2 to the human body will be useful not only to stimulate the formation of bones or cartilages, but also to improve the fibrosis and cirrhosis of kidneys, liver, lungs and heart depending on circumstances and administration conditions. Accordingly, the substantial scope of the present invention will be defined by the appended claims and equivalents thereof.

### INDUSTRIAL APPLICABILITY

As described in detail above, the present invention provides the non-activated polypeptides having pharmacological mechanisms completely different from those of the prior active proteins. The inventive TRPs have new mechanisms where they are in a non-activated state before in vivo administration but are activated in vivo. Thus, the inventive TRPs do not require additional equipment or cost for maintaining a three-dimensional structure to bind to the cell membrane receptor. In addition, the inventive TRPs are simple to produce, very easy to separate and purify, low in production cost, and also convenient to store, handle and administer. Accordingly, these TRPs can substitute for the prior activated proteins, such as rhBMPs.

1. A non-activated tissue-regeneration polypeptide (TRP) containing:
(a) a protein transduction domain (PTD) making the polypeptides to permeate cell membranes without cell membrane receptors;
(b) a furin activation domain (FAD) which has at least one proprotein convertase cleavage site and is cleaved by the proprotein convertase to activate tissue regeneration domain (TRD) in cells; and
(c) a non-activated TRD which is activated by the proprotein convertase cleavage of the FAD, and which stimulate the growth or formation of tissues or to induce the regeneration of tissues.

2. The non-activated TRP according to claim 1, wherein the proprotein convertase is furin.

3. The non-activated TRP according to claim 1, which has no three-dimensional stereoregularity and has no biological activity itself.

4. The non-activated TRP according to claim 1, wherein the proprotein convertase cleavage site of the FAD is cleaved by proprotein convertase present in living cells, whereby the TRD is activated, and the activated TRD is secreted extracellularly.

5. The non-activated TRP according to claim 1, wherein the TRD to be cleaved by proprotein convertase is selected from the group consisting of BMPs, TGF-β, β-NGF (β-nerve growth factor), β-amyloid, ADAMs (a disintergrin and metalloproteinase-like), TNF-α, MMPs (matrix metalloproteinases), and insulin-like growth factor (IGF-1).

6. The non-activated TRP according to claim 1, wherein the TRD is an amino acid sequence selected from the group consisting of SEQ ID NOs: 1 to 13.

7. The non-activated TRP according to claim 1, wherein the FAD is an amino acid sequence selected from the group consisting of SEQ ID NOs: 14 to 26.

8. The non-activated TRP according to claim 1, wherein the PTD is selected from the group consisting of TAT, drosophila melanogaster-derived Antp peptide, VP22 peptide and mph-1-btm.

9. The non-activated TRP according to claim 1, which is in the form of fusion polypeptide of PTD, FAD and TRD.

10. A recombinant vector inserted with an FAD-encoding base sequence in front of the 5′ region of TRD-encoding DNA, a PTD base sequence, a base sequence for tagging, and at least four histidine-encoding base sequences for separation and purification

11. A transformed bacteria with the recombinant vector of claim 10.

12. A method for preparing non-activated TRP, comprising the steps of:
(a) culturing the transformed bacteria of claim 11 to express a [PTD-FAD-TRD] polypeptide; and
(b) centrifuging the culture broth, and then removing the two-dimensional or three-dimensional structure of the polypeptide or converting the two-dimensional or three-dimensional structure to one-dimensional linear structure by addition of urea solution into the supernatant and cell pellet, and then purifying the [PTD-FAD-TRD] polypeptide.

13. The method for preparing non-activated TRP according to claim 12, wherein the TRD to be cleaved by proprotein convertase is selected from the group consisting of BMPs, TGF-β, β-NGF (β-nerve growth factor), P-amyloid, ADAMs (a disintergrin and metalloproteinase-like), TNF-α, MMPs (matrix metalloproteinases), and insulin-like growth factor (IGF-1).

14. The method for preparing non-activated TRP according to claim 12, wherein the TRD is an amino acid sequence selected from the group consisting of SEQ ID NOs: 1 to 13.

15. The method for preparing non-activated TRP according to claim 12, wherein the FAD is an amino acid sequence selected from the group consisting of SEQ ID NOs: 14 to 26.

16. The method for preparing non-activated TRP according to claim 12, wherein the PTD is selected from the group consisting of TAT, drosophila melanogaster-derived Antp peptide, VP22 peptide and mph-1-btm.

17. The method for preparing non-activated TRP according to claim 12, wherein the purification step comprises the sub-steps of binding the polypeptide to nickel-titanium beads, washing the beads with the same solution, and then eluting the beads with imidazole and a high-salt buffer solution.

18. A composition for stimulating the formation or regeneration of tissue, containing the non-activated TRP of claim 1, as an active ingredient.

19. The composition according to claim 18, wherein the tissue is bone or cartilage.

20. The composition according to claim 19, which further contains the growth factor selected from the group consisting of TGF-β, IGF, PDGF, and FGF.

21. A composition for improving the fibrosis or cirrhosis of organs, containing the non-activated TRP of claim 1, as an active ingredient.

22. The composition according to claim 21, which further contains the growth factor selected from the group consisting of TGF-β, IGF, PDGF, and FGF.
